(19) United States
(12) Patent Application Publication
(10) Pub. No.: US 2026/0306621 A1
(43) Pub. Date: Oct. 8, 2026

(54) DENGUE VIRUS SEROTYPE-SPECIFIC MODULATION OF T CELL RESPONSES

(71) Applicants: UNIVERSITY OF IOWA RESEARCH FOUNDATION, Iowa City, IA (US); THE UNITED STATES GOVERNMENT, AS REPRESENTED BY THE DEPARTMENT OF VETERANS AFFAIRS, Washington, DC (US)

(72) Inventors: Jack T. Stapleton, Iowa City, IA (US); Jinhua Xiang, Iowa City, IA (US); James McLinden, Iowa City, IA (US); Micaela Fosdick, Iowa City, IA (US)

(73) Assignees: UNIVERSITY OF IOWA RESEARCH FOUNDATION, Iowa City, IA (US); THE UNITED STATES GOVERNMENT, AS REPRESENTED BY THE DEPARTMENT OF VETERANS AFFAIRS, Washington, DC (US)

(21) Appl. No.: 19/485,134

(22) PCT Filed: May 17, 2024
(86) PCT No.: PCT/US2024/029999

§ 371 Date: Nov. 17, 2025

Related U.S. Application Data
(60) Provisional application No. 63/467,246, filed on May 17, 2023.

(51) Int. Cl. A61K 39/295; A61K 39/00; A61K 39/39; A61P 31/14; C07K 14/18

(52) U.S. Cl. CPC A61K 39/295; A61K 39/39; A61P 31/14; C07K 14/1825; A61K 2039/5254; A61K 2039/541; A61K 2039/55505; A61K 2039/55561; A61K 2039/55566; A61K 2039/55572; A61K 2039/55577; A61K 2039/70; C12N 2770/24122; C12N 2770/24134

(57) ABSTRACT

In certain embodiments, the present invention provides a recombinant Dengue virus (DENV) envelope (env) protein and compositions and uses of the DENV env proteins.

### CROSS-REFERENCE TO RELATED APPLICATION

This application claims priority to U.S. Provisional Application No. 63/467,246 that was filed on May 17, 2023, the entire contents of which are hereby incorporated by reference herein.

### STATEMENT REGARDING FEDERALLY SPONSORED RESEARCH

This invention was made with government support under Grant No. BX000207 awarded by the U.S. Department of Veterans Affairs. The U.S. government has certain rights in the invention.

### BACKGROUND

Dengue virus (DENV) infection is a major public health problem in tropical and sub-tropical regions of the world that causes approximately 400 million cases of DENV infection and 22,000 deaths annually. Despite this high incidence, DENV infection is categorized as a “neglected tropical disease.” DENV infection may cause a wide range of clinical manifestations, ranging from mild fever to severe dengue hemorragic fever (DHF) with thrombocytopenia, leucopenia, and increased vascular permeability and dengue shock syndrome (DSS).

DENV is a positive strand RNA virus that is classified within the Flavivirus genus of the Flaviviridae. Transmission of DENV occurs primarily from Aedes species mosquitoes to humans in urban or semi-urban settings. There are four distinct serotypes that are closely related, yet are antigenically different from each other (DENV1, DENV2, DENV3 and DENV-4). The four DENV serotypes may co-circulate in a geographic region, and many countries are hyper-endemic for all four serotypes.

The pathogenesis of Dengue (DEN) is incompletely understood. Although infection with one serotype appears to confer lifelong immunity to that serotype, cross-protection to the other three serotypes is only partial and temporary. Subsequent infection with a heterologous DENV serotype may occur, and in this setting, there is a marked increase in the risk of developing severe DEN. This increased risk is due in part because DENV infection with one serotype elicits cross-reacting but non-neutralizing antibodies to the other serotypes. These cross-reacting antibodies bind to heterotypic DENV during subsequent infection, and the resulting virus-antibody complex is recognized by Fc receptors on permissive cells leading to enhanced, antibody-dependent infection (ADE). ADE-associated severe disease complicates development of pan-serotype DENV vaccine approaches, as subsequent DENV infection was associated with more severe disease and hospitalization among vaccinated compared to non-vaccinated subjects in one study.

Although ADE is an important risk for DEN severity, serotypic differences in dengue severity have been documented for decades. Of note, serotypes 2 and 3 have been associated with more severe primary disease and in some studies, more frequent DEN hemorrhagic Fever (DHF) or DEN sepsis syndrome compared to Serotypes 1 and 4. No specific virological explanation for these serotypic differences in disease severity has been identified to date.

Many viruses, including several members of the Flavividae, interfere with T cell functions by altering expression of CD4 or by inhibiting various steps in the signaling cascade initiated by antigen engagement of the T cell receptor (TCR). It was previously found that DENV-2 envelope (env) protein does not inhibit TCR signaling when expressed in a Jurkat T cell line; however, investigation of the effect of other DENV serotypes on T cell functions have not been reported. Although DENV has been reported to infect peripheral blood mononuclear cells (PBMCs), the cell type(s) involved do not consistently support a role for T lymphocytes in DENV replication.

Accordingly, safe, effective vaccines against Dengue virus serotypes 1, 2, 3, and 4 (DENV-1, DENV-2, DENV-3 and DENV-4) are needed.

### SUMMARY

In an aspect, provided herein is a recombinant Dengue virus (DENV) envelope (env) protein, wherein the DENV is serotype 1 and amino acid 55 is not a valine, or wherein the DENV is serotype 4 and amino acid 55 is not a leucine.

In an aspect, provided herein is a composition comprising a protein comprising the recombinant DNEV envelope protein, wherein the DENV is serotype 1 and amino acid 55 is not a valine, or wherein the DENV is serotype 4 and amino acid 55 is not a leucine, and a pharmaceutically-acceptable, non-toxic vehicle, wherein the composition has enhanced antigenicity of DENV1 and DENV4 as compared to a non-recombinant DENV1 or DENV4 env protein.

In an aspect, provided herein is a recombinant Dengue virus (DENV) envelope (env) protein, wherein the DENV is serotype 2 or 3, and wherein the amino acid at position 55 is a valine or a leucine.

In an aspect, provided herein is a composition comprising the recombinant DENV envelope protein wherein the DENV is serotype 2 or 3, and wherein the amino acid at position 55 is a valine or a leucine, and a pharmaceutically-acceptable, non-toxic vehicle, wherein the composition has reduced inflammatory effects of DENV2 and DENV3 as compared to a non-recombinant DENV2 or DENV3 env protein.

In an aspect, provided herein is a method of protecting a susceptible patient against DENV infection comprising administering an effective amount of a composition described above to the patient in need thereof.

In an aspect, provided herein is a vaccine comprising a cell-adapted DENV, wherein the vaccine is effective in inducing immunological protection against DENV infection and is non-pathogenic.

In an aspect, provided herein is a vaccine comprising a biological agent or microbial component that is effective in inducing protection against DENV by stimulating a stronger cellular and humoral immune response to DENV as compared to a traditional DENV vaccine.

In an aspect, provided herein is a multivalent DENV vaccine comprising:
(a) an attenuated DENV1 virus,
(b) an attenuated DENV2 virus, wherein the envelope protein comprises a valine or a leucine at amino acid position 55,
(c) an attenuated DENV3 virus, wherein the envelope protein comprises a valine or a leucine at amino acid position 55, and
(d) an attenuated DENV4 virus.

In certain embodiments, the env protein of (b) or (c) further comprises a serine at amino acid 66.

In an aspect, provided herein is a multivalent DENV vaccine comprising:
(a) a subunit vaccine comprising a recombinant Dengue virus (DENV) envelope (env) protein, wherein the DENV is serotype 1 and amino acid 55 is not a valine,
(b) a subunit vaccine comprising a DENV2 env protein,
(c) a subunit vaccine comprising a DENV3 virus, and
(d) a subunit vaccine comprising a recombinant Dengue virus (DENV) envelope (env) protein, wherein the DENV is serotype 4 and amino acid 55 is not a leucine.

In certain embodiments, in (a) the amino acid at position 55 is threonine and wherein in (d) the amino acid at position 55 is threonine.

In certain embodiments, in (a) the amino acid at position 66 is threonine and wherein in (d) the amino acid at position 66 is threonine.

In an aspect, provided herein is a recombinant Dengue virus (DENV) comprising a recombinant envelope (env) protein, wherein the DENV replicates to a higher titer than wildtype virus.

### BRIEF DESCRIPTION OF DRAWINGS

FIG. 1. Dengue virus (DENV) serotypes 1 and 4 but not serotypes 2 and 3 inhibit TCR in a human T cell line (Jurkat E6.1). Jurkat cells were incubated with DENV 1-4, 229E coronavirus (229E), or no virus (Cell ctrl) prior to stimulation with anti-CD3 and soluble CD28 or no-stimulus (unstim) control. ***=p<0.01 vs. cell ctrl, DENV2, DENV 3, or 229E

FIG. 2. Dengue virus (DENV) serotypes 1 and 4 but not serotypes 2 and 3 inhibit TCR in primary human T cells. Healthy donor peripheral blood mononuclear cells were incubated with DENV 1-4, mumps virus (Mumps), yellow fever virus (YFV), or no virus (PBMCs) prior to stimulation with anti-CD3. IL-2 release from each donor was normalized to their cognate no-virus control. **=p<0.01 vs. cell ctrl, DENV2, DENV3, mumps, 229E

FIG. 3. Expression of dengue virus (DENV) serotypes 1 and 4 envelope but not serotypes 2 and 3 envelopes inhibit TCR in Jurkat T cell lines. IL-2 released by TCR stimulation in Jurkat cells expressing DENV 1-4 envelope proteins, the DENV1 envelope coding sequence with a frame-shift introduced to prevent expression of DV1 envelope protein (Den1 FS) or no protein (Cell ctrl) (PBMCw) prior to stimulation with anti-CD3 was measured. **=p<0.01 vs. cell ctrl, DENV2, DENV3, mumps, 229E

FIG. 4. Dengue virus (DENV) serotypes 1 and 4 envelope proteins, but not serotypes 2 and 3 inhibit TCR in primary human T cells. H293T cells expressing dengue virus envelope from serotypes 1, 2, 3, 4 (DENV1-4) or 293 coronavirus spike protein or no 293 T cells (PBMC stim) were plated onto the bottom of transwells. Healthy donor peripheral blood mononuclear cells (PBMCs; n=5) were added to the top well after 2 days of 293T cell growth, stimulated with anti-CD3 and IL-2 measured 16 hrs later. IL-2 release from each donor was normalized to their cognate no-virus control. **=p<0.01 vs. cell ctrl, DENV2, DENV3, mumps, 229E

FIG. 5. Dengue 1-3 serotype chimeric envelope proteins identify the region required for TCR inhibition. Replacing the first 65 amino acids of DENV3 envelope with DENV1 was sufficient to inhibit TCR signaling, and replacing the first 65 amino acids of DENV1 with DENV3 amino acids abolished the TCR inhibition.

FIG. 6. Position 55 in DENV envelope coding region critical in TCR signaling regulation. Expression of the peptide region from amino acid 49 to 62 in DENV1 was sufficient to inhibit TCR in Jurkat cells (and in 293 transwell system) and replacing the valine with the threonine present in DENV3 partially rescues the phenotype. Expressing the full DENV1 envelope with Y55T and S66T mutations (d1 env 55+66) rescues the TCR inhibition. Swapping the DENV1 amino acids into DENV3 full length env (T55V and T66S) makes the DENV3 env now inhibit TCR.

FIG. 7. Using reverse genetics, changing the DENV1 envelope sequence at amino acid 55 (valine) to the threonine present in DENV3 abolishes TCR signaling inhibition in replication-competent dengue virus.

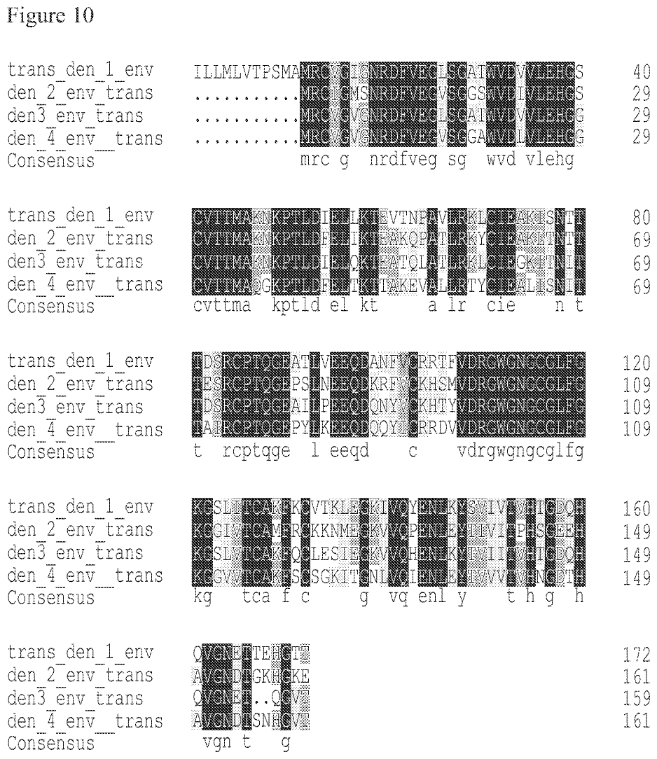

FIG. 8. A peptide of DENV1 amino acid 49-62 of the env protein is sufficient to inhibit signaling. It requires protein as the frameshift (FS) does not inhibit, and the valine as amino acid 55 (valine) is required. Changing this amino acid to the threonine present in DV2 and 3 reverses the T cell inhibition.

FIG. 9. Expression of the near-full length DENV1 and DENV3 envelope proteins (with C-terminal transmembrane sequence removed to enhance expression) shows that the valine amino acid 55 is required for inhibition of TCR signaling in DENV1 and replacement of DENV3 amino acid 55 and 66 with DENV1 amino acids (Valine and Serine) caused DV3 to inhibit TCR functions.

FIG. 10. A comparison of the sequences shows that there are only two amino acids (envelope amino acids 52 and 55) that are different between the 1-65 amino acid region in the envelope protein in serotypes 1 and 4, as compared to serotypes 2 and 3. Trans_den_1_env (SEQ ID NO:27); den_2_env_trans (SEQ ID NO:28); den3_env_trans (SEQ ID NO:29); den_4_env_trans (SEQ ID NO:30); consensus (SEQ ID NO:31).

FIGS. 11A-11D. Dengue virus (DENV) serotypes 1 and 4 but not serotypes 2 and 3 inhibit TCR in human PBMCs and a human T cell line (Jurkat E6.1). PBMCs from 5 donors (FIG. 11A) or Jurkat E6.1 cells (FIG. 11B) were incubated with DENV 1, 2, 3, 4 for 72 hrs prior to stimulation with anti-CD3 (PBMCs) or anti-CD3 and soluble CD28 (Jurkat cells), and IL-2 release into media measured 16 hrs later. Control cells with no virus incubation were stimulated (C-SC) or unstimulated (C-US) as indicated. PBMCs from three additional donors were incubated with DENV 1, 2, 3, 4 without (FIG. 11C), and following UV inactivation (FIG. 11D) with documented loss of infectivity. ANOVA identified significant variance in all four panels (p<0.01). **=p<0.01, ***=p<0.001 T test comparing each virus with stimulated control cell IL-2 results.

FIGS. 12A-12C. Abortive DENV infection in the Jurkat human T cell line. Equal amounts of RNA for each DENV serotype were added to Jurkat cells at 4° C. and incubated for 1 hr. Cells were washed ×3 with PBS and DENV RNA portion of total cellular RNA measured, or warmed to 37° C. for 1 hr prior to quantifying DENV RNA (FIG. 12A). To determine if bound virus after warming reflected virions adherent to cells without entry, cells were washed again and treated with trypsin (0.25%) for 5 minutes, washed, and viral RNA quantified (FIG. 12A). Trypsin treated cells were maintained at 37° C. in media for 3 days, and intracellular (FIG. 12B) or supernatant (FIG. 12C) DENV RNA concentrations were determined on days 0, 1, 2, and 3. **=p<0.01 compared to DENV 1.

FIGS. 13A-13D. DENV envelope protein (env) expression effects on T cell receptor signaling. The four DENV serotype envelopes are schematically shown in FIG. 13A, and FS represents DENV 1 env coding sequence in which a frame-shift mutation was inserted to abolish protein expression. Jurkat cell TCR-mediated IL-2 release was inhibited for DENV1 and 4, but not DENV 2 and 3, or the DENV 1 FS cells (FIG. 13B). Control Jurkat cells stimulated with anti-CD3/CD28=C-SC. Chimeric viruses are shown in FIG. 13C. The N-terminal 133 amino acids of DENV 1 were required for TCR inhibition (FIG. 13D). ***=P<0.001.

FIG. 14. Characterization of DENV 1 coding region and critical amino acids required for TCR inhibition. Jurkat cell lines were generated that expressed DENV 1 env amino acids 1-133, 1-120, 1-80, and 1-65. Further truncations found that amino acids 49-62 were sufficient to inhibit TCR (graph and bars 1, 2, 4-8 and 10). Substitution of DENV 2 and 3 threonine at position 55 restored TCR function in both 1-133 and 49-72 constructs (bars 3, 9 and 11). Protein expression was required, as introduction of a frameshift at the start of aa 45-72 (FS) abolished TCR inhibition. C-SC=Jurkat Cell stimulated control. ***=p<0.001 vs. C-SC

FIG. 15. Synthetic peptides inhibited TCR-mediated IL-2 release. Human PBMCs were incubated with 100, 50 and 10 mg/mL DENV 1 peptide representing envelope aas 49-62 with or without HIV TAT protein transduction domain for 24 hr prior to stimulation with anti-CD3. Peptides with substitution of threonine for valine at position 55 were tested (V55T). IL-2 release was measured 16 hrs later. C-SC=Cell stimulated control. ***=p<0.001 vs. C-SC

FIGS. 16A-16B. Recombinant DENV 1, 2, 3, 4 and DENV 1 mutants' effect on TCR. rDENV 1, 2, 3, and 4 viruses were generated using CPER, and following detection of cytopathic effect in Huh 7.5 cells, the second pass supernatant was characterized. All four rDENV had detectable env protein intracellularly and released into cell culture media using immunoblot analysis (FIG. 16A). Addition of rDENV 1, 2, 3, 4 and the rDENV 1 V55T, S66T, and double mutant to Jurkat cells prior to stimulation with anti-CD3/CD28 was consistent with prior studies. rDENV 1 and 4 inhibited TCR-mediated IL-2 release while rDENV 2 and 3 did not. Further, the single rDENV 1 V55T was sufficient to reverse TCR inhibition while S66T was not (FIG. 16B). **=p<0.01 vs C-SC, ***=p<0.001 vs. C-SC.

FIG. 17. Effect of DENV env mutations on TCR signaling in primary T cells. rDENV viruses and mutants were generated by CPER and mutations characterized by sequence analysis. Following incubation with 1×10⁶ PBMCs (MOI=1) for 1 hrs, cells were stimulated with anti-CD3 and IL-2 in culture supernatants measured. C-SC=control stimulated cells. *=p<0.05, **=p<0.01, ***=p<0.001.

FIGS. 18A-18D. Effect of DENV env mutations on viral replication in Hub 7.5 cells and Vero cells. rDENV 1 with native envelope or with V55T, S66T, or both were used to infect Huh 7.5 cells (FIG. 18A) or Vero cells (FIG. 18B) using an MOI of 1. Similarly, rDENV 3 with native, T55V, T66S, or both were used to infect Huh 7.5 cells (FIG. 18C) or Vero cells (FIG. 18D). Virus in culture supernatant was titered on Day 0, 2, 4, and 6 post-infection as Indicated. **=p<0.01 vs. native, ***=p<0.001 vs native.

### DETAILED DESCRIPTION

There are four serotypes of dengue virus (DENV1, DENV2, DENV3, and DENV4) that differ primarily by their relative neutralization by antibodies raised against the other types. The severity of dengue virus appears to be increased in serotypes 2 and 3 with primary infection. The most severe dengue virus infections occur in people who have had an initial infection with one serotype, who have subsequent infections with different serotypes. This severe dengue causes dengue hemorrhagic fever (DHF) or dengue shock syndrome (DSS). The presence of antibodies to the initial dengue serotype that cross-react but do not neutralize the second infection results in enhancement of infection of cells expressing Fc receptors, and this is one factor that contributes to DHF/DSS. For years, it has been known that serotypes 2 and 3 are more likely to cause DHF and DSS than serotypes 1 and 4; however, there are currently no published explanations for these serotype differences. Of note, live attenuated dengue vaccines are limited due to the increased risk of complications in those with pre-existing dengue antibodies.

Severe DENV is mediated by over-exuberant immune response. It is now believed that the lack of T-cell Receptor (TCR) inhibition by DENV2 and DENV3 explains the increased pathogenesis both with primary and secondary DENV.

The present data show that DENV serotypes 1 and 4 (DENV1 and DENV4) interfere with T cell signaling through the T cell receptor. In contrast, DENV serotypes 2 and 3 (DENV2 and DENV3) do not interfere with T cell signaling. Mapping experiments with chimeric proteins defined the T cell inhibitory region to amino acids 1-65 of the envelope protein. Comparison of sequences show only two amino acids (envelope amino acids 52 and 55) that are different between the 1-65 amino acid region in the envelope protein in serotypes 1 and 4 as compared to serotypes 2 and 3. Replacement of the first 65 amino acids of DENV3 envelope protein into the DENV1 envelope protein abolishes the T cell inhibitory effect, indicating that this region is required for T cell dysfunction and accounts for the immunomodulatory phenotype.

Following identification of the amino acids involved, DENV2 and DENV3 viruses are generated that interfere with T cell functions and their pathogenicity is examined in an animal model as potential candidate vaccines. DENV1 and DENV4 envelope proteins are generated that do not interfere with T cell function to see if their immunogenicity is significantly enhanced for consideration of subunit vaccine candidates. Because severe Dengue and dengue hemorrhagic fever are associated with DENV serotypes 2 and 3, and candidate live-attenuated vaccines cause severe adverse effects in those with prior infection (usually DENV2), this finding offers an opportunity to modify the live-attenuated vaccine strains towards safer forms. Further, mutation of this amino acid in DENV1 and DENV4 to the version that does not interfere with T cell function enhances the immunogenicity of subunit (non-infectious) DENV1 and DENV4 vaccines.

The goal of the present work was to determine if the immunopotency of DENV1 and DENV4 envelopes could be enhanced by mutating these stereotypes to no longer reduce TCR signaling. Another goal was to determine of the severity could be reduced by mutating live-attenuated DENV vaccine strains to inhibit TCR signaling in DENV serotypes 2 and 3.

The effects of DENV1, DENV2, DENV3, and DENV4 virus and their cognate viral envelope proteins on T cell receptor (TCR) signaling and T cell functions were examined. It was found that DENV1 and DENV4 interfered with TCR signaling while DENV2 and DENV3 did not. These data suggest that serotypic differences in envelope protein sequences may contribute to the increased severity and risk for DHF and DSS observed for DENV2 and DENV3 compared to DENV1 and DENV4.

Certain aspects of the present invention provide a recombinant Dengue virus (DENV) envelope (env) protein, wherein the DENV is serotype 1 and amino acid 55 is not a valine, or wherein the DENV is serotype 4 and amino acid 55 is not a leucine.

In certain aspects, the amino acid at position 55 is threonine.

In certain aspects, the protein further comprises a threonine at amino acid 66 of the DENV env protein.

In certain aspects, the DENV is serotype 1.

In certain aspects, the DENV is serotype 4.

Certain aspects of the present invention provide a composition comprising a protein comprising the recombinant DNEV envelope protein, wherein the DENV is serotype 1 and amino acid 55 is not a valine, or wherein the DENV is serotype 4 and amino acid 55 is not a leucine, and a pharmaceutically-acceptable, non-toxic vehicle, wherein the composition has enhanced antigenicity of DENV1 and DENV4 as compared to a non-recombinant DENV1 or DENV4 env protein.

In certain aspects, the DENV env protein further comprises a threonine at amino acid 66.

In certain aspects, the DENV is serotype 1.

In certain aspects, the DENV is serotype 4.

In certain aspects, the composition further comprises an effective amount of an immunological adjuvant.

In certain aspects, the immunological adjuvant is selected from the group consisting of an aluminium salts Monophosphoryl lipid A (MPL) and aluminum salt, monophosphoryl lipid A (MPL) and aluminum salt, and an oil in water emulsion comprising squalene, monophosphoryl lipid A (MPL) and QS-21 and cytosine phosphoguanine (CpG).

Certain aspects of the present invention provide a recombinant Dengue virus (DENV) envelope (env) protein, wherein the DENV is serotype 2 or 3, and wherein the amino acid at position 55 is a valine or a leucine.

In certain aspects, the amino acid at position 55 of the DENV env protein is a valine.

In certain aspects, the amino acid at position 55 of the DENV env protein is a leucine.

In certain aspects, the DENV is serotype 2.

In certain aspects, the DENV is serotype 3.

In certain aspects, the DENV env protein further comprises a serine at amino acid 66.

Certain aspects of the present invention provide a composition comprising the recombinant DENV envelope protein wherein the amino acid at position 55 is a valine, wherein the DENV is serotype 2 or 3, and a pharmaceutically-acceptable, non-toxic vehicle, wherein the composition has reduced inflammatory effects of DENV2 and DENV3 as compared to a non-recombinant DENV2 or DENV3 env protein.

In certain aspects, the DENV is serotype 2.

In certain aspects, the DENV is serotype 3.

In certain aspects, the DENV env protein further comprises a serine at amino acid 66.

In certain aspects, the composition further comprises an effective amount of an immunological adjuvant.

In certain aspects, the immunological adjuvant is selected from the group consisting of an aluminium salts Monophosphoryl lipid A (MPL) and aluminum salt, monophosphoryl lipid A (MPL) and aluminum salt, and an oil in water emulsion comprising squalene, monophosphoryl lipid A (MPL) and QS-21 and cytosine phosphoguanine (CpG).

Certain aspects of the present invention provide a method of protecting a susceptible patient against DENV infection comprising administering an effective amount of a composition described above to the patient in need thereof.

In certain aspects, the composition is administered by intramuscular, intradermal, subcutaneous delivery, or via a mucosal surface.

In certain aspects, the composition is administered by subcutaneous or intramuscular injection.

Certain aspects of the present invention provide a vaccine comprising a cell-adapted DENV, wherein the vaccine is effective in inducing immunological protection against DENV infection and is non-pathogenic.

In certain aspects, the DENV is serotype 2 or 3.

Certain aspects of the present invention provide a vaccine comprising a biological agent or microbial component that is effective in inducing protection against DENV by stimulating a stronger cellular and humoral immune response to DENV as compared to a traditional DENV vaccine.

In certain aspects, the DENV is serotype 1 or 4.

In an aspect, provided herein is a multivalent DENV vaccine comprising:
(a) a subunit vaccine comprising a recombinant Dengue virus (DENV) envelope (env) protein, wherein the DENV is serotype 1 and amino acid 55 is not a valine,
(b) a subunit vaccine comprising a DENV2 env protein,
(c) a subunit vaccine comprising a DENV3 virus, and
(d) a subunit vaccine comprising a recombinant Dengue virus (DENV) envelope (env) protein, wherein the DENV is serotype 4 and amino acid 55 is not a leucine.

In certain embodiments, in (a) the amino acid at position 55 is threonine and wherein in (d) the amino acid at position 55 is threonine.

In certain embodiments, in (a) the amino acid at position 66 is threonine and wherein in (d) the amino acid at position 66 is threonine.

In an aspect, provided herein is a multivalent DENV vaccine comprising:
(a) an attenuated DENV1 virus,
(b) an attenuated DENV2 virus, wherein the envelope protein comprises a valine or a leucine at amino acid position 55,
(c) an attenuated DENV3 virus, wherein the envelope protein comprises a valine or a leucine at amino acid position 55, and
(d) an attenuated DENV4 virus.

In certain embodiments, the env protein of (b) or (c) further comprises a serine at amino acid 66

In an aspect, provided herein is a recombinant Dengue virus (DENV) comprising a recombinant envelope (env) protein, wherein the DENV replicates to a higher titer than wildtype virus.

In certain embodiments, the DENV is serotype 1 and amino acid 55 of the env protein is not a valine, or wherein the DENV is serotype 4 and amino acid 55 of the env protein is not a leucine.

In certain embodiments, the amino acid at position 55 is threonine.

In certain embodiments, the amino acid at position 66 is threonine.

In certain embodiments, the titer is greater than 1.00 log₁₀ as compared to the wildtype virus titer. In certain embodiments, the titer is greater than 2.00 log₁₀ as compared to the wildtype virus titer. In certain embodiments, the titer is greater than 1.0, 1.1, 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, or 3.0 log₁₀.

### DENV Envelope Protein

The DENV envelope proteins from serotypes 1-4 have the following amino acid sequences (minimal sequence inhibiting for DENV1 and not inhibiting for DENV3 in bold below in the full 1-133 sequence):

DENV1
(SEQ ID NO: 1)
MRCVGIGNRDFVEGLSGATWVDVVLEHGSCVTTMAKNKPTLDIELLKTEVTNPAVLRKLCIEAK
ISNTTTDSRCPTQGEATLVEEQDANFVCRRTFVDRGWGNGCGLFGKGSLITCAKFKCVTKLEGK
IVQYENL
(SEQ ID NO: 2)
MRCVGIGNRDFVEGLSGATWVDVVLEHGSCVTTMAKNKPTLDIELLKTE VTNPAVLRKLCIEA
KISNTTTDSRCPTQGEATLVEEQDANFVCRRTFVDRGWGNGCGLFGKGSLITCAKFKCVTKLEG
KIVQYENL
DENV2
(SEQ ID NO: 3)
MRCIGMSNRDFVEGVSGGSWVDIVLEHGSCVTTMAKNKPTLDFELIKTEAKQPATLRKYCIEAK
LTNTTTESRCPTQGEPSLNEEQDKRFVCKHSMVDRGWGNGCGLFGKGGIVTCAMFRCKKNMEGK
VVQPENL
(SEQ ID NO: 4)
MRCIGMSNRDFVEGVSGGSWVDIVLEHGSCVTTMAKNKPTLDFELIKTE AKQPATLRKYCIEA
KLTNTTTESRCPTQGEPSLNEEQDKRFVCKHSMVDRGWGNGCGLFGKGGIVTCAMFRCKKNMEG
KVVQPENL
DENV3
(SEQ ID NO: 5)
MRCIGVGNRDFVEGLSGATWVDVVLEHGGCVTTMAKNKPTLDIELQKTEATQLATLRKLCIEGK
ITNITTDSRCPTQGEAILPEEQDQNYVCKHTYVDRGWGNGCGLFGKGSLVTCAKFQCLESIEGK
VVQHENL
(SEQ ID NO: 6)
MRCIGVGNRDFVEGLSGATWVDVVLEHGGCVTTMAKNKPTLDIELQKTE ATQLATLRKLCIEG
KITNITTDSRCPTQGEAILPEEQDQNYVCKHTYVDRGWGNGCGLFGKGSLVTCAKFQCLESIEG
KVVQHENL
DENV4
(SEQ ID NO: 7)
MRCVGVGNRDFVEGVSGGAWVDLVLEHGGCVTTMAQGKPTLDFELTKTTAKEVALLRTYCIEAL
ISNITTATRCPTQGEPYLKEEQDQQYICRRDVVDRGWGNGCGLFGKGGVVTCAKFSCSGKITGN
LVQIENL
(SEQ ID NO: 8)
MRCVGVGNRDFVEGVSGGAWVDLVLEHGGCVTTMAQGKPTLDFELTKTT AKEVALLRTYCIEA
LISNITTATRCPTQGEPYLKEEQDQQYICRRDVVDRGWGNGCGLFGKGGVVTCAKFSCSGKITG
NLVQIENL
1
(SEQ ID NO: 9)
MRCVGIGNRD FVEGLSGATW VDVVLEHGSC VTTMAKNKPT LDIELLKTEV
TNPAVLRKLC IEAKISNTTT DSRCPTQGEA TLVEEQDANF VCRRTFVDRG
WGNGCGLFGK GSLITCAKFK CVTKLEGKIV QYE
2.
(SEQ ID NO: 10)
MRCIGMSNRD FVEGVSGGSW VDIVLEHGSC VTTMAKNKPT LDFELIKTEA
KQPATLRKYC IEAKLTNTTT ESRCPTQGEP SLNEEQDKRF VCKHSMVDRG
WGNGCGLFGK GGIVTCAMFR CKKNMEGKVV QPE
3.
(SEQ ID NO: 11)
MRCIGVGNRD FVEGLSGATW VDVVLEHGGC VTTMAKNKPT LDIELQKTEA
TQLATLRKLC IEGKITNITT DSRCPTQGEA ILPEEQDQNY VCKHTYVDRG
WGNGCGLFGK GSLVTCAKFQ CLESIEGKVV QHE
4.
(SEQ ID NO: 12)
MRCVGVGNRD FVEGVSGGAW VDLVLEHGGC VTTMAQGKPT LDFELTKTTA
KEVALLRTYC IEALISNITT ATRCPTQGEP YLKEEQDQQY ICRRDVVDRG
WGNGCGLFGK GGVVTCAKFS CSGKITGNLV QIE

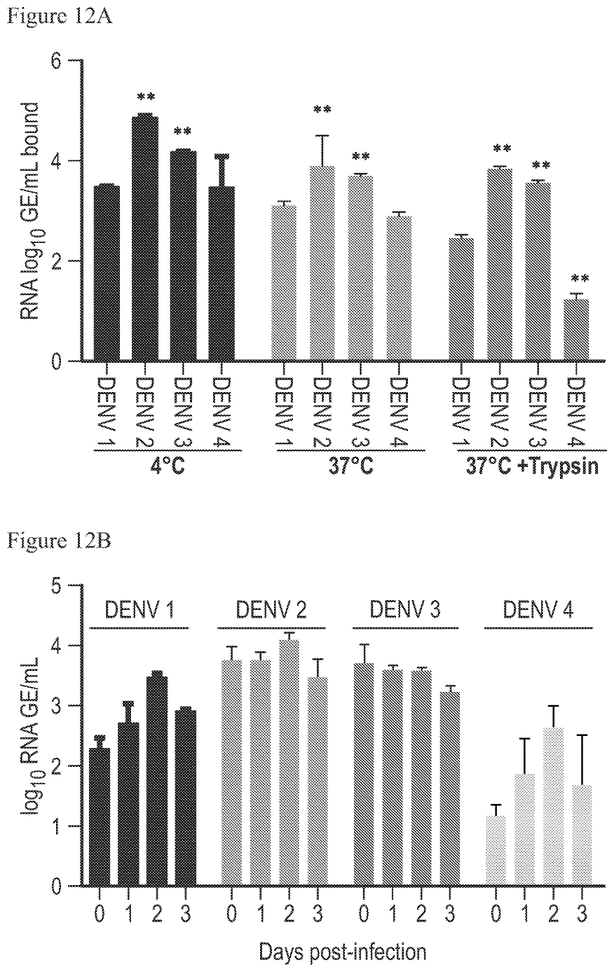

A comparison of the sequences shows that there are only two amino acids (envelope amino acids 52 and 55) that are different between the 1-65 amino acid region in the envelope protein in serotypes 1 and 4, as compared to serotypes 2 and 3. FIG. 10.

TABLE 1
Amino acids in DENV 1 and 4 env that differ
from those in DENV 2 and 3

| AA # | DENV1 | DENV2 | DENV3 | DENV4 |
|---|---|---|---|---|
| 52 | N | Q | Q | E |
| 55 | V | T | T | L |
| 66 | S | T | T | S |
| 81 | T | S | I | Y |
| 83 | V | N | P | K |
| 93 | R | K | K | R |
| 94 | R | H | H | R |
| 96 | F | M | Y | V |
| 120 | K | R | Q | S |
| 122 | V | Q | L | S |
| 123 | T | K | E | G |
| 124 | K | S | S | N |
| 129 | I | L | V | I |
| 132 | Y | P | H | I |

52 N/E-Q, 55 V/L-T, 66 S-T, 81 all 4 T/S/I/Y, 83 all4 V/N/P/K, 93 R-K, 94 R-H, 96 all 4 F/M/Y/V, 120 all 4 K/R/Q/S, 122 all 4 V/Q/L/S, 123 T/K/E/G, 124 K-N/S, 129 I/L-V, 132 all 4 Y/P/H/I

In certain aspects the DENV envelope protein is conjugated or linked to another peptide or to a polysaccharide. For example, immunogenic proteins well-known in the art, also known as “carriers,” may be employed. Useful immunogenic proteins include keyhole limpet hemocyanin (KLH), bovine serum albumin (BSA), ovalbumin, human serum albumin, human gamma globulin, chicken immunoglobulin G and bovine gamma globulin. Useful immunogenic polysaccharides include polysaccharides from other pathogens, such as those that are effective as vaccines. The immunogenic polysaccharides or proteins of other pathogens can be conjugated to, linked to, or mixed with DENV envelope protein.

The terms “protein,” “peptide” and “polypeptide” are used interchangeably herein.

The term “amino acid” includes the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in Dextrorotary or Levorotary stereoisomeric forms, as well as unnatural amino acids (e.g., phosphoserine, phosphothreonine, phosphotyrosine, hydroxyproline, and gamma-carboxyglutamate; hippuric acid, octahydroindole-2-carboxylic acid, statine, 1,2,3,4,-tetrahydroisoquinoline-3-carboxylic acid, penicillamine, ornithine, □itrulline, alpha-methyl-alanine, para-benzoylphenylalanine, phenylglycine, propargylglycine, sarcosine, and tert-butylglycine). The term also comprises natural and unnatural amino acids (Dextrorotary and Levorotary stereoisomers) bearing a conventional amino protecting group (e.g. acetyl or benzyloxycarbonyl), as well as natural and unnatural amino acids protected at the carboxy terminus (e.g., as a (C₁-C₆)alkyl, phenyl or benzyl ester or amide; or as an α-methylbenzyl amide). Other suitable amino and carboxy protecting groups are known to those skilled in the art (See for example, Greene, T. W.; Wutz, P. G. M., Protecting Groups In Organic Synthesis; second edition, 1991, New York, John Wiley & sons, Inc, and documents cited therein). An amino acid can be linked to the remainder of a compound through the carboxy terminus, the amino terminus, or through any other convenient point of attachment, such as, for example, through the sulfur of cysteine.

The invention encompasses isolated or substantially purified protein compositions. In the context of the present invention, an “isolated” or “purified” polypeptide is a polypeptide that exists apart from its native environment and is therefore not a product of nature. A polypeptide may exist in a purified form or may exist in a non-native environment such as, for example, a transgenic host cell. For example, an “isolated” or “purified” protein, or biologically active portion thereof, is substantially free of other cellular material, or culture medium when produced by recombinant techniques, or substantially free of chemical precursors or other chemicals when chemically synthesized. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed proteins or partial-length proteins encoded thereby are also encompassed by the present invention. By “fragment” or “portion” is meant a full length or less than full length of the amino acid sequence of, a polypeptide or protein.

A “variant” of a molecule is a sequence that is substantially similar to the sequence of the native molecule.

“Isolated” means altered or removed from the natural state. For example, a nucleic acid or a peptide naturally present in a living animal is not “isolated.” But the same nucleic acid or peptide partially or completely separated from the coexisting materials of its natural state is “isolated.” An isolated nucleic acid or protein can exist in substantially purified form, or can exist in a non-native environment such as, for example, a host cell. Unless it is particularly specified otherwise herein, the proteins, virion complexes, antibodies and other biological molecules forming the subject matter of the present invention are isolated, or can be isolated.

The term “substantial identity” in the context of a protein indicates that a protein comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to a reference sequence over a specified comparison window. Optimal alignment is conducted using the homology alignment algorithm of Needleman and Wunsch, J. Mol. Biol. 48:443 (1970). An indication that two protein sequences are substantially identical is that one protein is immunologically reactive with antibodies raised against the second protein. Thus, a protein is substantially identical to a second protein, for example, where the two proteins differ only by a conservative substitution.

### Adjuvants

The term “adjuvant” as used herein refers to non-specific stimulators of the immune response or substances that allow generation of a depot in the host, which when combined with the vaccine and pharmaceutical composition, respectively, of the present invention may provide for an even more enhanced immune response. Vaccines commonly contain two components: antigen (e.g., DENV envelope protein) and adjuvant. The antigen is the molecular structure encoded by the pathogen or tumor against which the immune response is directed. To activate an antigen-specific immune response, the antigen must be presented in the appropriate immunostimulatory microenvironment. In certain embodiments, adjuvants establish such microenvironments by stimulating the production of immune-activating molecules such as proinflammatory cytokines. Vaccine efficacy depends on the types of antigen and adjuvant, and how they are administered. Striking the right balance among these components is important to eliciting the desired immunological result.

Immunogenic compositions as described herein also comprise, in certain embodiments, one or more adjuvants. An adjuvant is a substance that enhances the immune response when administered together with an immunogen or antigen. A number of cytokines or lymphokines have been shown to have immune modulating activity, and thus are useful as adjuvants, including, but not limited to, the interleukins 1-α, 1-β, 2, 4, 5, 6, 7, 8, 10, 12, 13, 14, 15, 16, 17 and 18 (and its mutant forms); the interferons-α, β and γ; granulocyte-macrophage colony stimulating factor (GM-CSF); macrophage colony stimulating factor (M-CSF); granulocyte colony stimulating factor (G-CSF); and the tumor necrosis factors α and β. Still other adjuvants that are useful with the immunogenic compositions described herein include chemokines, including without limitation, MCP-1, MIP-1α, MIP-1β, and RANTES; adhesion molecules, such as a selectin, e.g., L-selectin, P-selectin and E-selectin; mucin-like molecules, e.g., CD34, GlyCAM-1 and MadCAM-1; a member of the integrin family such as LFA-1, VLA-1, Mac-1 and p150.95; a member of the immunoglobulin superfamily such as PECAM, ICAMs, e.g., ICAM-1, ICAM-2 and ICAM-3, CD2 and LFA-3; co-stimulatory molecules such as CD40 and CD40L; growth factors including vascular growth factor, nerve growth factor, fibroblast growth factor, epidermal growth factor, B7.2, PDGF, BL-1, and vascular endothelial growth factor; receptor molecules including Fas, TNF receptor, Flt, Apo-1, p55, WSL-1, DR3, TRAMP, Apo-3, AIR, LARD, NGRF, DR4, DR5, KILLER, TRAIL-R2, TRICK2, and DR6; and Caspase (ICE).

Still other adjuvants include muramyl peptides, such as N-acetyl-muramyl-L-threonyl-D-isoglutamine (thr-MDP), N-acetyl-normuramyl-L-alanine-2-(1′-2′ dipalmitoyl-sn-glycero-3-hydroxyphosphoryloxy)-ethylamine (MTP-PE); oil-in-water emulsions, such as MF59 (containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton, MA)), and SAF (containing 10% Squalene, 0.4% Tween 80, 5% pluronic-blocked polymer L121, and thr-MDP, either microfluidized into a submicron emulsion or vortexed to generate a larger particle size emulsion); aluminum salts (alum), such as aluminum hydroxide, aluminum phosphate, aluminum sulfate; Amphigen; Avridine; L121/squalene; D-lactide-polylactide/glycoside; pluronic polyols; killed Bordetella; saponins, such as Stimulon™ QS-21 (Antigenics, Framingham, MA.), ISCOMATRIX (CSL Limited, Parkville, Australia), and immunostimulating complexes (ISCOMS); Mycobacterium tuberculosis; bacterial lipopolysaccharides; synthetic polynucleotides such as oligonucleotides containing a CpG motif; IC-31 (Intercell AG, Vienna, Austria); a pertussis toxin (PT) or mutant thereof, a cholera toxin or mutant thereof, or an E. coli heat-labile toxin (LT) or mutant thereof, particularly LT-K63, LT-R72.

Suitable adjuvants used to enhance an immune response further include, without limitation, MPL™ (3-O-deacylated monophosphoryl lipid A, Corixa, Hamilton, MT). Also suitable for use as adjuvants are synthetic lipid A analogs or aminoalkyl glucosamine phosphate compounds (AGP), or derivatives or analogs thereof, which are available from Corixa (Hamilton, MT). One such AGP is 2-[(R)-3-Tetradecanoyloxytetradecanoylamino]ethyl 2-Deoxy-4-O-phosphono-3-O—[(R)-3-tetradecanoyoxytetradecanoyl]-2-[(R)-3-tetradecanoyloxytetradecanoyl-amino]-b-D-glucopyranoside, which is also known as 529 (formerly known as RC529). This 529 adjuvant is formulated as an aqueous form (AF) or as a stable emulsion (SE).

Suitable adjuvants include but are not limited to surfactants, e.g., hexadecylamine, octadecylamine, lysolecithin, dimethyldioctadecylammonium bromide, N,N-dioctadecyl-N′—N-bis(2-hydroxyethyl-propane di-amine), methoxyhexadecyl-glycerol, and pluronic polyols; polanions, e.g., pyran, dextran sulfate, poly IC, polyacrylic acid, carbopol; peptides, e.g., muramyl dipeptide, MPL, aimethylglycine, tuftsin, oil emulsions, alum, and mixtures thereof. Other potential adjuvants include the B peptide subunits of E. coli heat labile toxin or of the cholera toxin. Finally, the immunogenic product may be incorporated into liposomes for use in a vaccine formulation, or may be conjugated to proteins such as keyhole limpet hemocyanin (KLH) or human serum albumin (HSA) or other polymers.

In certain aspects, the adjuvant is an aluminium salt, Monophosphoryl lipid A (MPL) and aluminum salt, monophosphoryl lipid A (MPL) and aluminum salt, or an oil in water emulsion comprising squalene, monophosphoryl lipid A (MPL) and QS-21 and cytosine phosphoguanine (CpG).

### Nucleic Acids, Expression Cassettes and Vectors Encoding DENV Env Proteins

In some embodiments, the polypeptides described herein are prepared using recombinant methods. Accordingly, certain embodiments provide polynucleotides (e.g., isolated polynucleotides) comprising a nucleic acid sequence encoding any of the polypeptides described herein. The polynucleotides may be single-stranded or double-stranded. In some embodiments, the polynucleotide is DNA. In some embodiments, the polynucleotide is cDNA. In some embodiments, the polynucleotide is RNA.

In certain embodiments, the nucleic acid further comprises a promoter.

Certain embodiments of the invention provide an expression cassette comprising a nucleic acid sequence described herein and a promoter operably linked to the nucleic acid.

In certain embodiments, the promoter is a regulatable promoter. In certain embodiments, the promoter is a constitutive promoter.

In certain embodiments, the expression cassette further comprises an expression control sequence (e.g., an enhancer) operably linked to the nucleic acid sequence. Expression control sequences and techniques for operably linking sequences together are well known in the art.

Nucleic acids/expression cassettes encoding a polypeptide described herein can be engineered into a vector using standard ligation techniques, such as those described in Sambrook and Russell, Molecular Cloning: A Laboratory Manual, Cold Spring Harbor Laboratory Press Cold Spring Harbor, NY (2001). For example, ligations can be accomplished in 20 mM Tris-Cl pH 7.5, 10 mM MgCl2, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration).

Accordingly, certain embodiments of the invention provide a vector comprising an expression cassette described herein. In particular, certain embodiments provide a vector comprising an expression cassette comprising a promoter operably linked to a nucleic acid sequence encoding a polypeptide of the invention.

Non-limiting examples of vectors include plasmids and viral expression systems, such as a lentiviral, adenoviral, and adeno-associated virus (AAV) expression systems. Further non-limiting examples mammalian expression vectors include the pRc/CMV, pSV2gpt, pSV2neo, pcDNA3, pcDNAI/amp, pcDNAI/neo, pSV2-dhfr, pMSG, pSVT7, pTk2, pRSVneo, pko-neo, and pHyg-derived vectors. In certain embodiments, the vector is a lentivirus vector. In certain embodiments, the vector is a vector described herein.

### Vaccines and Therapeutic Agents

In certain embodiments, the present invention provides vaccines for use to protect mammals against or to treat an DENV infection.

In certain embodiments, the present invention provides therapeutic agents that inhibit DENV infection.

The present invention provides a DENV vaccine that is an attenuated DENV, wherein the vaccine is effective in inducing immunological protection against DENV infection and is non-pathogenic. As used herein the term “attenuated” is defined as being rendered less virulent in a non-chemically-induced or genetically engineered manner. For example, “attenuated” may mean passaged in tissue culture. The term “non-pathogenic” is used herein to mean non-virulent or unable to induce illness. The term “cell-adapted” is defined herein to mean virus that is transferred or passaged from one culture of cells to the next culture of cells.

The present invention provides a vaccine that contains a biological agent or microbial component that is effective in inducing improved protection against DENV by stimulating a strong cellular response in addition to a strong humoral immune response as compared to traditional DENV vaccines. The live biological agent of the vaccine may be a virus. For example the virus may be an attenuated virus, a recombinant virus or a virus that has been altered by chemical, physical or molecular means. The terms “traditional” or “conventional” are used to refer to currently available DENV vaccines. The new DENV vaccine of the present invention is different from conventional vaccines because the new vaccine is selected on its ability to generate anti-DENV cellular immunity. The conventional vaccines are selected on their ability to generate humoral immunity without an assessment of their ability to generate cellular immunity.

The present invention also provides a method of protecting a patient by administering to the patient an immunologically protective amount of a vaccine of the present invention. As used herein, the term “immunologically protective” means that the vaccine is effective in inducing a protective immune response. An immunological response to a composition or vaccine is the development in the host of a cellular and/or antibody-mediated immune response to the polypeptide or vaccine of interest. Usually, such a response consists of the subject producing antibodies, B cell, helper T cells, suppressor T cells, and/or cytotoxic T cells directed specifically to an antigen or antigens included in the composition or vaccine of interest.

The vaccine may be prepared by attenuating the virus in a culture of antigen-presenting cells, such as macrophages, B cells or dendritic cells. The virus may be passaged one or more times until the virus has a low level of virulence, but still retains immunoprotective properties.

As used herein, the term “therapeutic agent” or “therapeutic complex” refers to any agent or material that has a beneficial effect on the subject recipient. Thus, “therapeutic agent” embraces both therapeutic and prophylactic molecules having nucleic acid or protein components.

To prepare a vaccine, the DENV envelope protein can be isolated, lyophilized and stabilized. The DENV envelope protein may then be adjusted to an appropriate concentration, optionally combined with a suitable vaccine adjuvant, and packaged for use.

In certain embodiments, the vaccine is a recombinant cell that expresses an DENV envelope protein.

In certain embodiments, the DENV envelope protein, or recombinant cell that expresses an DENV envelope protein is adjusted to an appropriate concentration and can be formulated with any suitable adjuvant, diluent, pharmaceutically acceptable carrier, or any combination thereof. As used herein the phrase “pharmaceutically acceptable carrier” is intended to include any and all solvents, dispersion media, coatings, antibacterial and antifungal agents, isotonic and absorption delaying agents, excipients and the like, compatible with pharmaceutical administration. The use of such media and agents for pharmaceutically active substances is well known in the art. Physiologically acceptable vehicles may be used as carriers and/or diluents. A pharmaceutically acceptable vehicle is understood to designate a compound or a combination of compounds entering into a pharmaceutical or immunogenic composition which does not cause side effects, and which makes it possible, for example, to facilitate the administration of the active compound, to increase its life and/or its efficacy in the body, to increase its solubility in solution or alternatively to enhance its preservation. These pharmaceutically acceptable vehicles are well known and will be adapted by persons skilled in the art according to the nature and the mode of administration of the active compound chosen. These include, but are not limited to, water, Ringer's solution, an appropriate isotonic medium, glycerol, ethanol and other conventional solvents, phosphate buffered saline, and the like.

“Treating” as used herein refers to ameliorating at least one symptom of, curing and/or preventing the development of a given disease or condition.

“Antigen” refers to a molecule capable of being bound by an antibody. An antigen is additionally capable of being recognized by the immune system and/or being capable of inducing a humoral immune response and/or cellular immune response leading to the activation of B- and/or T-lymphocytes. An antigen can have one or more epitopes (B- and/or T-cell epitopes). Antigens as used herein may also be mixtures of several individual antigens. “Antigenic determinant” refers to that portion of an antigen that is specifically recognized by either B- or T-lymphocytes. B-lymphocytes responding to antigenic determinants produce antibodies, whereas T-lymphocytes respond to antigenic determinants by proliferation and establishment of effector functions critical for the mediation of cellular and/or humoral immunity.

An “immune response” refers to a humoral immune response and/or cellular immune response leading to the activation or proliferation of B- and/or T-lymphocytes and/or and antigen presenting cells. In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent. An “immunogenic polypeptide” is a polypeptide that elicits a cellular and/or humoral immune response, whether alone or linked to a carrier. Preferably, antigen presenting cell may be activated.

A substance that “enhances” an immune response refers to a substance in which an immune response is observed that is greater or intensified or deviated in any way with the addition of the substance when compared to the same immune response measured without the addition of the substance. For example, the lytic activity of cytotoxic T lymphocytes (CTLs) can be measured, e.g. using a ⁵¹Cr release assay, in samples obtained with and without the use of the substance during immunization. The amount of the substance at which the CTL lytic activity is enhanced as compared to the CTL lytic activity without the substance is said to be an amount sufficient to enhance the immune response of the animal to the antigen. In certain embodiments, the immune response in enhanced by a factor of at least about 2, such as by a factor of about 3 or more. The amount or type of cytokines secreted may also be altered. Alternatively, the amount of antibodies induced or their subclasses may be altered.

The terms “immunize,” “immunization” or related terms refer to conferring the ability to mount a substantial immune response (comprising antibodies and/or cellular immunity such as effector CTL) against a target antigen or epitope. These terms do not require that complete immunity be created, but rather that an immune response be produced which is substantially greater than baseline. For example, a mammal may be considered to be immunized against a target antigen if the cellular and/or humoral immune response to the target antigen occurs following the application of methods of the invention.

The term “immunotherapeutic” refers to a composition for the treatment of diseases, disorders or conditions. More specifically, the term is used to refer to a method of treatment wherein a beneficial immune response is generated by vaccination or by transfer of immune molecules. An “immunologically effective amount” refers to an amount of a composition sufficient to induce an immune response in an individual when introduced into that individual. In the context of active immunization, the term is synonymous with “immunogenically effective amount.” The amount of a composition necessary to be immunologically effective varies according many factors including to the composition, the presence of other components in the composition, the antigen, the route of immunization, the individual, the prior immune or physiologic state etc.

The term “epitope” refers to basic element or smallest unit of recognition by an individual antibody or T-cell receptor, and thus the particular domain, region or molecular structure to which said antibody or T-cell receptor binds. An antigen may consist of numerous epitopes while a hapten, typically, may possess few epitopes. As used herein “correspond essentially to” refers to an epitope that will elicit an immunological response at least substantially equivalent to the response generated by the native epitope. An immunological response to a composition or vaccine is the development in the host of a cellular and/or antibody-mediated immune response to the polypeptide or vaccine of interest. Usually, such a response consists of the subject producing antibodies, B cell, helper T cells, suppressor T cells, and/or cytotoxic T cells directed specifically to an antigen or antigens included in the composition or vaccine of interest.

Vaccines of the present invention can also include effective amounts of immunological adjuvants, known to enhance an immune response. An “effective amount” refers to an amount necessary or sufficient to realize a desired biologic effect. An effective amount of the composition would be the amount that achieves this selected result, and such an amount could be determined as a matter of routine by a person skilled in the art. For example, an effective amount for treating an immune system deficiency could be that amount necessary to cause activation of the immune system, resulting in the development of an antigen specific immune response upon exposure to antigen. The term is also synonymous with “sufficient amount.” The effective amount for any particular application can vary depending on such factors as the disease or condition being treated, the particular composition being administered, the size of the subject, and/or the severity of the disease or condition. One of ordinary skill in the art can empirically determine the effective amount of a particular composition of the present invention without necessitating undue experimentation.

### Formulations and Methods of Administration

In certain embodiments, an effective amount of the vaccine or therapeutic agent is administered to the subject. “Effective amount” refers to an amount of a compound, formulation, material, or composition, as described herein effective to achieve a particular biological result. Such results may include, but are not limited to the prevention or inhibition of gonococcal infection as determined by any means suitable in the art.

In certain embodiments, an amount of the vaccine is administered in order to immunize to the subject. As used herein, “immunization” or “vaccination” are used interchangeably herein and are intended for prophylactic or therapeutic immunization or vaccination.

To immunize a subject, the composition is administered parenterally, usually by intramuscular or subcutaneous injection in an appropriate vehicle. In certain embodiments, the vaccine is administered subcutaneously or via a mucosal surface, such as an oral, intranasal or intradermal surface. In certain embodiments, the vaccine is administered by using infusion techniques.

Vaccine formulations will contain an effective amount of the active ingredient in a vehicle, the effective amount being readily determined by one skilled in the art. The active ingredient may typically range from about 1% to about 95% (w/w) of the composition, or even higher or lower if appropriate. The quantity to be administered depends upon factors such as the age, weight and physical condition of the animal or the human subject considered for vaccination. The quantity also depends upon the capacity of the animal's immune system to synthesize antibodies, and the degree of protection desired. Effective dosages can be readily established by one of ordinary skill in the art through routine trials establishing dose response curves. The subject is immunized by administration of the biofilm peptide or fragment thereof in one or more doses. Multiple doses may be administered as is required to maintain a state of immunity to the bacterium of interest.

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

To prepare a vaccine, the purified composition can be isolated, lyophilized and stabilized. The composition may then be adjusted to an appropriate concentration, optionally combined with a suitable vaccine adjuvant, and packaged for use.

In certain embodiments, “pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions. For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Additional ingredients such as fragrances or antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

To immunize a subject, the DENV envelope protein is administered parenterally, usually by intramuscular or subcutaneous injection in an appropriate vehicle. Other modes of administration, however, such as oral delivery or intranasal delivery, are also acceptable. Vaccine formulations will contain an effective amount of the active ingredient in a vehicle.

Formulations will contain an effective amount of the active ingredient in a vehicle, the effective amount being readily determined by one skilled in the art. “Effective amount” is meant to indicate the quantity of a compound necessary or sufficient to realize a desired biologic effect. The active ingredient may typically range from about 1% to about 95% (w/w) of the composition, or even higher or lower if appropriate. The amount for any particular application can vary depending on such factors as the severity of the condition. The quantity to be administered depends upon factors such as the age, weight and physical condition of the animal considered for vaccination and kind of concurrent treatment, if any. The quantity also depends upon the capacity of the animal's immune system to synthesize antibodies, and the degree of protection desired. Typically, dosages used in vitro may provide useful guidance in the amounts useful for in situ administration of the composition. Additionally, effective dosages can be readily established by one of ordinary skill in the art through routine trials establishing dose response curves. The subject is immunized by administration of the composition thereof in one or more doses. Multiple doses may be administered as is required to maintain a state of immunity to the target. For example, the initial dose may be followed up with a booster dosage after a period of about four weeks to enhance the immunogenic response. Further booster dosages may also be administered. The composition may be administered multiple (e.g., 2, 3, 4 or 5) times at an interval of, e.g., about 1, 2, 3, 4, 5, 6 or 7, 14, or 21 days apart.

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

Thus, the present compositions may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the present compositions may be combined with one or more excipients and used in the form of ingestible tablets, buccal tablets, troches, capsules, elixirs, suspensions, syrups, wafers, and the like. Such preparations should contain at least 0.1% of the present composition. The percentage of the compositions may, of course, be varied and may conveniently be between about 2 to about 60% of the weight of a given unit dosage form. The amount of present composition in such therapeutically useful preparations is such that an effective dosage level will be obtained.

Useful dosages of the compositions of the present invention can be determined by comparing their in vitro activity, and in vivo activity in animal models. The amount of the compositions described herein required for use in treatment will vary with the route of administration and the age and condition of the subject and will be ultimately at the discretion of the attendant veterinarian or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### Example 1

### Serotype-Specific Inhibition of TCR-Mediated Activation

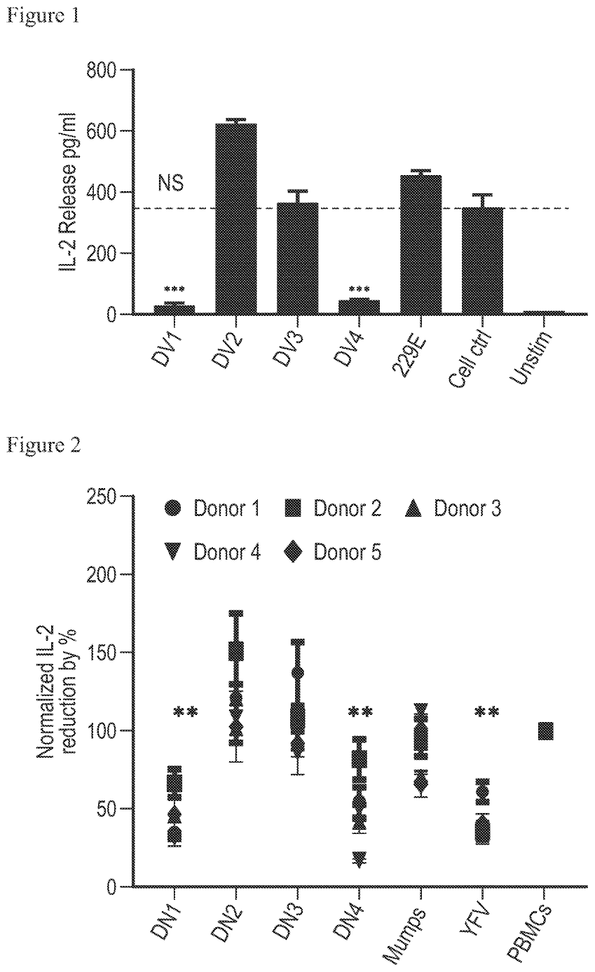

To examine the effect of DENV serotypes 1, 2, 3 and 4 on T cell functions, primary human PBMCs from five healthy blood donors were incubated with each DV serotype (MOI=1) 37° C. in triplicate, following which TCR was stimulated with anti-CD3 and soluble CD28 for 16 hrs. IL-2 release was significantly reduced in human T cell lines (Jurkat, HUT78) incubated in DENV1 and DENV4 compared to control cells without DENV incubation. In contrast, DENV2 and DENV3 did not reduce IL-2 release compared to controls (FIG. 1).

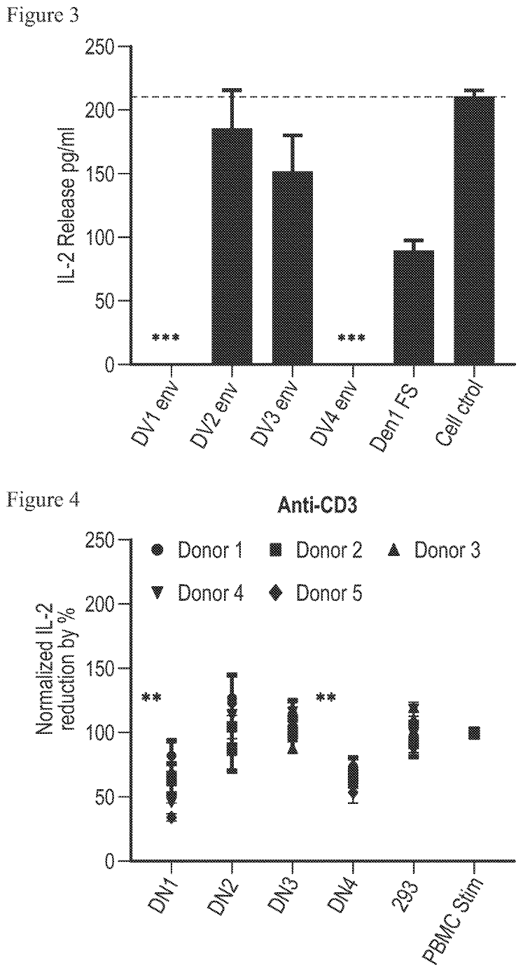

The same pattern of TCR inhibition by DENV1 and DENV4 but not DENV2 and DENV3 was observed in primary human T cells (FIG. 2). Experiments were performed in triplicate in five individual healthy donors.

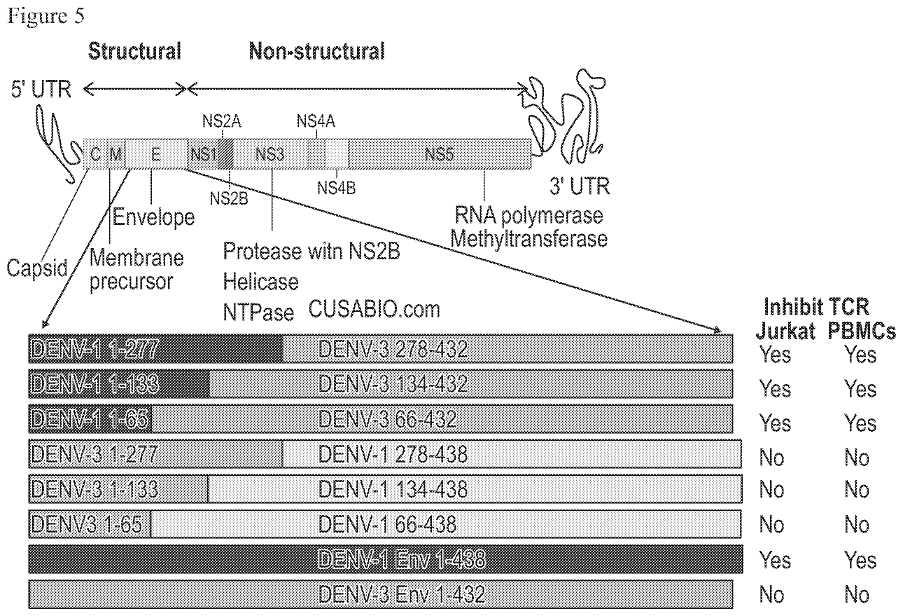

To determine if DENV1-4 bound to and entered primary or Jurkat human T cells, DENV1-4 were applied to PBMCs, maintained at 4° C. for 1 hr, washed extensively, and viral RNA bound to the cells was measured. No differences in DENV binding to T cells was observed (FIG. 3).

### Example 2

### DENV Envelope Protein Mediates TCR Inhibition

Since viral replication was not required for TCR inhibition, and DENV1 and DENV4 particles were sufficient to inhibit TCR, it was postulated that viral structural proteins may be involved in TCR inhibition. DENV1, 2, 3, 4 envelope proteins (env) were expressed, where each contained a C-terminal influenza hemagglutinin epitope in Jurkat cells and 293T cells. Expression of full-length env proteins was detected in 293T cells but not Jurkat cells. Deletion of the DENV1-4 envelopes C-terminal transmembrane domain resulted in detection of expression in Jurkat cells.

Stimulating TCR with anti-CD3/CD28 in Jurkat cells expressing the DENV1-4 envelope protein confirmed that DENV1 and 4 inhibit TCR whereas DENV2, DENV3 and 229E do not (FIG. 3). This required protein expression, as Jurkat cells expressing the DENV1 env coding sequence in which a frameshift mutation was included to prevent protein expression did not result in TCR inhibition.

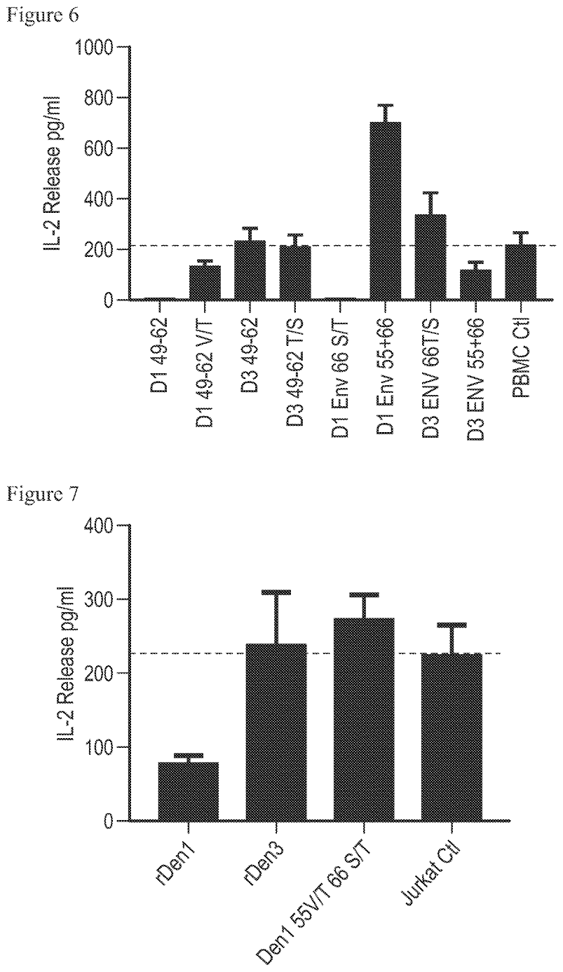

In addition to this system, the DENV envelopes were expressed in 293T cells, applied to the bottom of 24 well transwell plates, and primary healthy donor PBMCs were added to the upper well of the transwells after 2 days incubation in the 293T cells. DENV1 and 4 envelopes inhibited TCR-mediated IL-2 release in the PBMCs while DENV2 and 3 did not (FIG. 4).

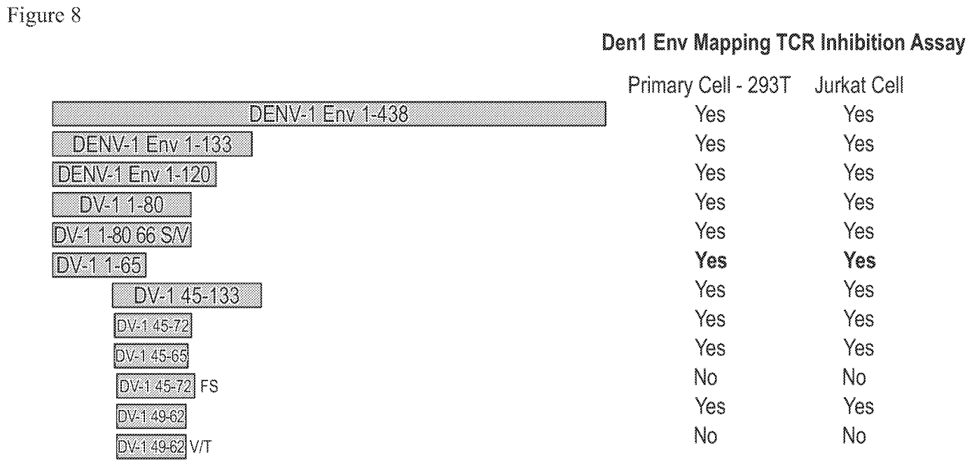

To map the region involved, chimeric DENV1 and DENV3 envelopes were generated. FIG. 5 illustrates the DENV1-DENV3 chimeras generated, and IL-2 release data is summarized in the figure. The first 65 amino acids of DENV1 were sufficient to maintain the TCR inhibitory phenotype.

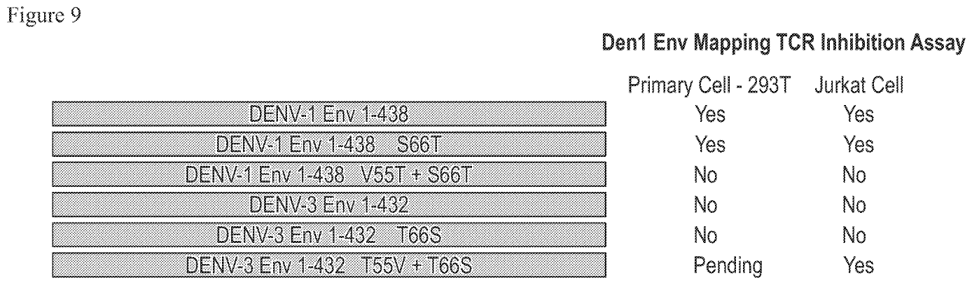

Comparison of DENV1-4 sequences identified only 1 amino acid in the first 65 amino acids of the envelope coding region (position 55) that are different in both DENV1 and 4 from both DENV2 and 3. Expression of amino acids 49 to 62 of DENV1 inhibited TCR and DENV3 did not. The DENV1 valine at 55 was replaced with the threonine present in DENV3 with rescue of the TCR inhibition. Further, insertion of a valine at position 55 in DENV3 resulted in TCR inhibition (FIG. 6).

Using the CPER method, recombinant DENV1 and 3 were generated and the TCR inhibition was consistent with earlier DENV studies. Changing the Valine at env position 55 to the threonine in DENV3 reversed the TCR signaling inhibition. The complimentary mutations in DENV3 are in progress.

In summary, DENV2 and DENV3 cause more severe disease including hemorrhagic fever and dengue shock syndrome in people with prior dengue infection with a heterologous serotype. This is immunologically mediated. Some live attenuated vaccines have had similar problems. By mutating DENV2 and DENV3 to inhibit TCR, this will reduce risk of hyper-immune responses in vaccine recipients in live-attenuated vaccine use.

Subunit flavivirus envelope-based vaccines have not proven very immunogenic. This is likely influenced by the T cell inhibitory effects noted in DENV1 and DENV4. By removing these TCR inhibitory motifs in recombinant proteins, these envelope proteins are more immunogenic and thus more effective in subunit vaccine approaches.

### Example 3

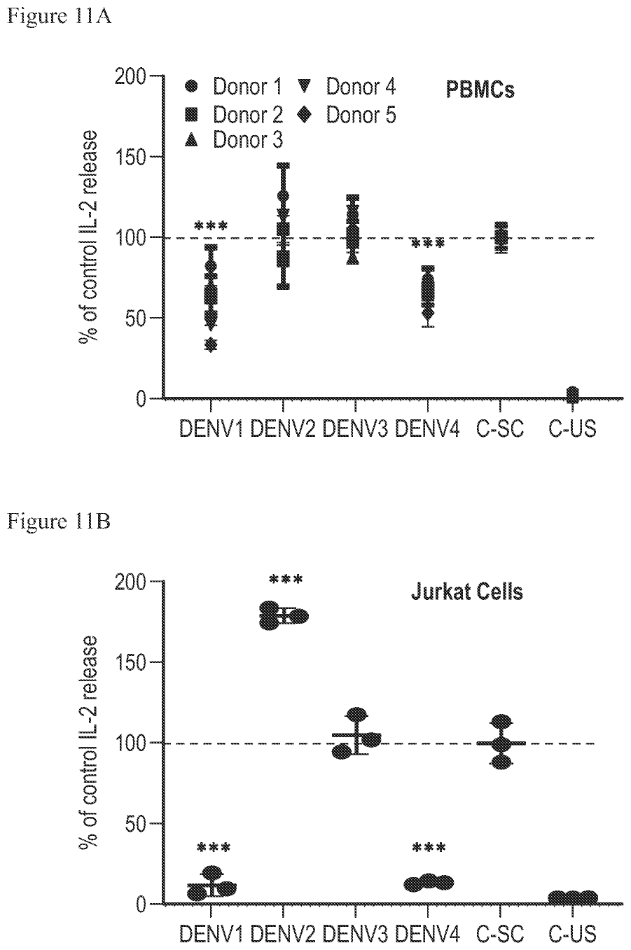

A peptide of DENV1 env protein amino acids 49-62 is sufficient to inhibit signaling. FIG. 8. It requires protein as the frameshift (FS) does not inhibit, and the valine as amino acid 55 (valine) is required. Changing this amino acid to the threonine present in DENV2 and DENV3 reverses the T cell inhibition.

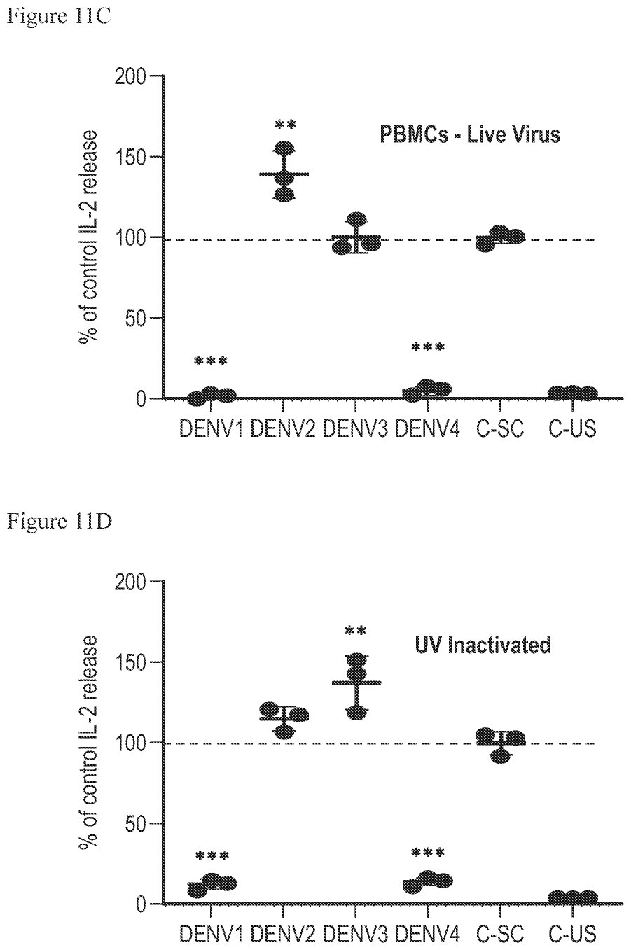

Expression of the near-full length DENV1 and DENV3 envelope proteins (with C-terminal transmembrane sequence removed to enhance expression) showed that the valine amino acid 55 is required for inhibition of TCR signaling in DENV1 and replacement of DENV3 amino acids 55 and 66 with DENV1 amino acids (Valine and Serine) caused DV3 to inhibit TCR functions. FIG. 9.

### Example 4

### Dengue Virus Serotypes 1 and 4 Inhibit TCR Via Specific Amino Acids in the Envelope Protein and these Amino Acids can be Modulated to Enhance Production

The effect of DENV-1, DENV-2, DENV-3, and DENV-4 virus and their cognate viral env proteins on T cell infection, and the effect of DENV serotypes on TCR signaling was examined. It was found that DENV-1 and DENV-4 interfered with TCR signaling while DENV-2 and DENV-3 did not. Specific amino acid differences involved in these different effects were characterized, and the effect of these amino acids on viral replication were examined. The data suggest that serotypic differences in env protein sequences influence serotype-specific replication and immune interference through the TCR, which may contribute to the increased severity and risk for DHF observed for DENV-2 and DENV-3 compared to DENV-1 and DENV-4.

### Materials and Methods

Dengue viruses: DENV 1 through 4 viruses (Isolates 16007, 16681, 16562, and 1036, GenBank AF180817, NC_001474, U11673, and MW793460, respectively) were kindly provided by Dr. James Brien (University of Kentucky). In this example, these four isolates are referred to as DENV 1, 2, 3, or 4. Plasmids containing the complete DENV 1 through 4 genome sequences of clinical isolates and the linker plasmid containing the CMV promoter and delta ribozyme were used in circular polymerase extension reactions to generate infectious virus as previously described were kindly provided by Dr. Alex Ploss. The four isolates representing DENV serotypes 1, 2, 3, and 4 used in these studies were GenBank OK605756, OK605758, OK605762, and OK605767, respectively. These viruses are referred to as rDENV 1, 2, 3, or 4 to clarify that they represent CPER generated viruses.

Viral RNA replication was detected and quantified by real-time PCR using 3′UTR primers for DENV 1, 2, and 3 sense: 5′-GAR AGA CCA GAG ATC CTG CTG TCT-3′ (SEQ ID NO: 13), antisense: 5′-ACC ATT CCA TTT TCT GGC GTT-3′ (SEQ ID NO:14), and probe 5′-/56-FAM/AGC ATC ATT CCA GGC AC/3IABKFQ/-3′ (SEQ ID NO:15). For DENV 4, M coding region primers were utilized: sense 5′-GCT GGT GCA ATC TCA CGT CTA-3′ (SEQ ID NO:16), antisense 5′-GCG CGA ATC CTG GGT TT-3′ (SEQ ID NO:17), and probe 5′-/56-FAM/ATG CAC CCA GAG CGG AGA ACG GA/3IABKFQ/-3′ (SEQ ID NO: 18). Viral infectivity was determined by measuring the TCID₅₀ in Vero cells as described for YFV.

CPER: Recombinant DENV (rDENV) were generated by ligation of overlapping PCR fragments covering the entire dengue virus genome and overlapping with a linker product containing a CMV promoter upstream of the viral genome start, and with a delta ribozyme following the authentic genome 3′UTR sequence. Circular DNA polymerase (TAKARA R050B) was used to amplify the ligated overlapping PCR products and the mixture was used to transfect both Huh 7.5 cells and Vero cells using Xtremegene™ reagent (Roche 6366236001). Cells were seeded in 6 wells plate with 2 ml of completed DMEM 10% FCS, 1% Pen/Strep and 1% Glutamine. The day before transfection, 200 ul OptiMEM, 25 μl CPER product and 6 μl Xtremegene were mixed and incubated at RT for 15 mins prior to adding the mixture dropwise to the well. Cells were cultured 12-16 hours in the incubator, washed once with warm media, maintained culture with 2% FCS media, and monitored for evidence of viral cytopathic effect. Culture supernatant fluids obtained 5 to 7 days post transfection were used to infect new cells to generate a stock virus preparation (passage 2 or P2). P2 virus was used to characterize viral replication in different cell types, determine the effect of different serotypes on TCR signaling, and to validate env protein expression. P2 supernatants were also used to extract RNA for env-specific RT-PCR spanning regions of mutagenesis to confirm amino acid sequences in recombinant viruses.

Cells: DENV infections were propagated in human cell lines (Huh 7.5 and 293T), a hamster cell line (BHK 21), a mosquto cell line (C6/36), and an African Green Monkey cell line (Vero) in DMEM containing 10% FCS, 1% Pen/Strep and 1% Glutamine.

For T cell signaling studies, primary human peripheral blood mononuclear cells (PBMCs) and Jurkat cells were employed. For PBMCs, the study was approved by the University of Iowa Institutional Review Board, and all subjects provided written informed consent. Following written informed consent, blood was obtained from subjects with no history of HIV, hepatitis B or C virus infection or immune suppressive medication use. PBM were prepared from blood obtained from healthy donors within 4 hours of acquisition, and all studies utilized fresh cells that had not been frozen. For TCR stimulation studies, PBMCs (1×10⁶ cells/ml) were incubated with plate-bound anti-CD3 (200 ng/ml, OKT3 clone, eBioscience™) and IL-2 released into cell culture superatants was quantified 16 hours post-stimulation using human IL-2 ELISA kit (BD Biosciences™) according to the manufacturer's instructions. Each experiment was performed in triplicate.

The Effect of Dengue virus envelope protein expression on TCR-mediated IL-2 release: Tet-off Jurkat or 293T cell lines expressing the complete DENV 1, 2, 3, 4 env proteins and a variety of chimeric DENV 1 and 3 or truncated DENV envs were generated. DENV env protein coding sequences were amplified from viral RNA by RT-PCR using primers designed to place a BamH1 restriction site at the 5′-end of each env coding region in the sense primer, and a Not1 site at the 5′ end of the antisense primer for ligation into the expression vector. Specific primers employed for DENV 1, DENV 2, DENV 3, and DENV 4 were the following:

1)
sense
(SEQ ID NO: 19)
5′-AGGATCCCATGCGATGCGTGGGAATAG-3′,
antisense
(SEQ ID NO: 20)
5′-AGCGGCCGCTGCCTGAACCATGACTCC-3′,
2) sense
(SEQ ID NO: 21)
5′-AGGATCCCATGCGTTGCATAGGAATGTC-3′,
antisense
(SEQ ID NO: 22)
5′-AGCGGCCGCGGCCTGCACCATGACTC-3′,
3)
(SEQ ID NO: 23)
5′-AGGATCCCATGAGATGTGTAGGAGTAGG-3′,
antisense
(SEQ ID NO: 24)
5′-AGCGGCCGCAGCTTGCACCACGGCTC 3′,
and
4)
(SEQ ID NO: 25)
5′-AGGATCCCATGCGATGCGTAGGAGTAG-3′,
antisense
(SEQ ID NO: 26)
5′-AGCGGCCGCTTGAACCGTGAAGCC-3′.

Products were digested with BamH1 and Not1 and ligated into a modified pTRE2-HGY plasmid (Clontech™, Inc.) that generates a bicistronic message in which the DENV env sequences were followed by the encephalomyocarditis virus (EMC) internal ribosomal entry site (IRES) directing translation of eGFPmclind. Jurkat (tet-off) and 293T (tet-off) cell lines (Clontech™, Inc) were transfected (Nucleofector® II, Lonza™ Inc.) and cell lines selected for resistance to hygromycin and neomycin. GFP positive cells were bulk sorted using a BD FACS Diva™ (University of Iowa Flow Cytometry Facility). Protein expression was analyzed by measuring GFP by flow cytometry (BD® LSR II) and by immunoblot using antibodies directed against a C-terminal histidine tag (Qiagen®). Insert sequences were confirmed by sequencing DNA from stably expressing cell lines (University of Iowa DNA Core Facility).

To assess TCR-mediated IL-2 release from the Jurkat CD4+ T cell line, cells (5×10⁶ cells/ml) were stimulated with plate-bound anti-CD3 (5 μg/ml, OKT3 clone, eBioscience™) and soluble CD28 antibody (5 μg/ml, clone CD28.2, BD® Biosciences) unless stated otherwise. Following 24 hours of stimulation, IL-2 release were measured by ELISA. To assess the effect of DENV recombinant proteins on primary T cells, 293T cells expressing the various DENV env protein constructs (or 293T cells without DENV sequences) were plated onto the lower well of 24-well Transwell™ plates (Corning®, Inc; 1×10⁵/well). PBMCs were added to the top chamber (1×10⁶/well) 48 hrs later maintaining the 293T conditioned media, and following 48 hr incubation, PBMCs were stimulated with anti-CD3 (200 ng/ml). IL-2 release was measured in supernatant media 16 hrs later by Quantikine® ELISA kit (R&D™ Systems) according to manufacturer's instructions.

Immunoblot Analysis: Cells (1×10⁶) were lysed using cell lysis buffer (Cell Signaling), followed by sonication. Lysates were separated by polyacrylamide gel electrophoresis and transferred to nitrocellulose membranes (BIORAD™). Membranes were incubated in protein-free blocking buffer (Thermo Scientific™) for 1 hour at room temperature followed by incubation with primary antibodies. Immunoreactive proteins were detected with Amersham™ ECL™ (GE Healthcare™) using an Invitrogen™ iBright™ 1500 System Imager. Primary antibodies used were: anti-HA and anti-Dengue Env antibodies (Invitrogen™, Inc., Catalog #26183 and MA 1-82795, respectively), and anti-actin (Sigma™ catalog #A2066). Immunoblots were quantified by ImageJ™.

Statistics: Data represent the average of three technical replicates unless otherwise stated, and error bars are provided to show the standard deviation. Each triplicate experiment was independently performed at least twice with consistent results. For experiments utilizing primary human PBMCs, at least three different healthy blood donors were studied. For presenting data from PBMCs, data were normalized to the average of the triplicate control value. Control value variation from average are also shown. All statistical analyses were performed using GraphPad software V4.0 (GraphPad™ Software Inc.). Two-sided Student's t test was used to compare results between test and controls. One-way analysis of variance (ANOVA) was used to determine whether differences between the means of three or more independent groups. P values less than 0.05 were considered statistically significant.

### Results

### Differences in DENV Serotype Inhibition of TCR-Mediated IL-2 Release

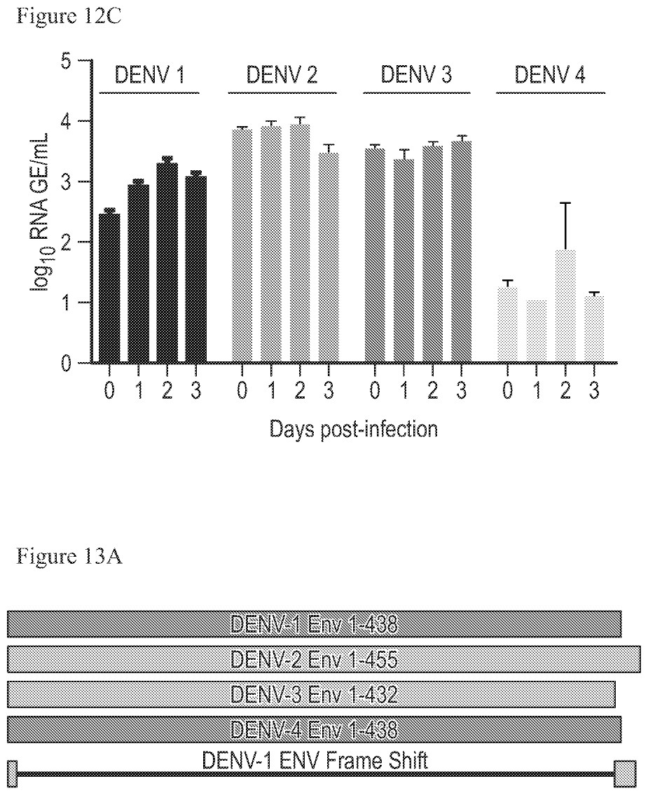

To examine the effect of DENV serotypes 1 through 4 on T cell functions, primary human PBMCs from five healthy blood donors were incubated with each DENV serotype (MOI=1) for 72 hrs at 37° C. in triplicate, following which TCR was stimulated with anti-CD3 for 16 hrs. FIG. 11A shows the average IL-2 release for each donor for stimulated control (no virus) cells, unstimulated control cells, and for cells incubated in DENV 1, 2, 3, or 4. Although the magnitude of inhibition varied between donors, DENV 1 and 4 statistically and consistently reduced IL-2 release compared to stimulated control cells and cells incubated with DENV 2 and 3 prior to anti-CD3 stimulation. Similarly, Jurkat E6.1 cells incubated with DENV 1 and 4 had significantly less IL-2 released compared to control cells and cells incubated in DENV 2 and 3 following anti-CD3 stimulation with the difference that soluble CD28 was included in stimulation as described (FIG. 11B). Data from the Jurkat cell experiments represent the average of triplicate experiments from three independent experiments. Testing DENV 1, 2, 3, and 4 with and without UV-inactivation demonstrated that viral replication was not required for DENV 1 and 4 TCR inhibition (FIGS. 11C, 11D).

### DENV Inoculation of Human T Cells Leads to Abortive Infection

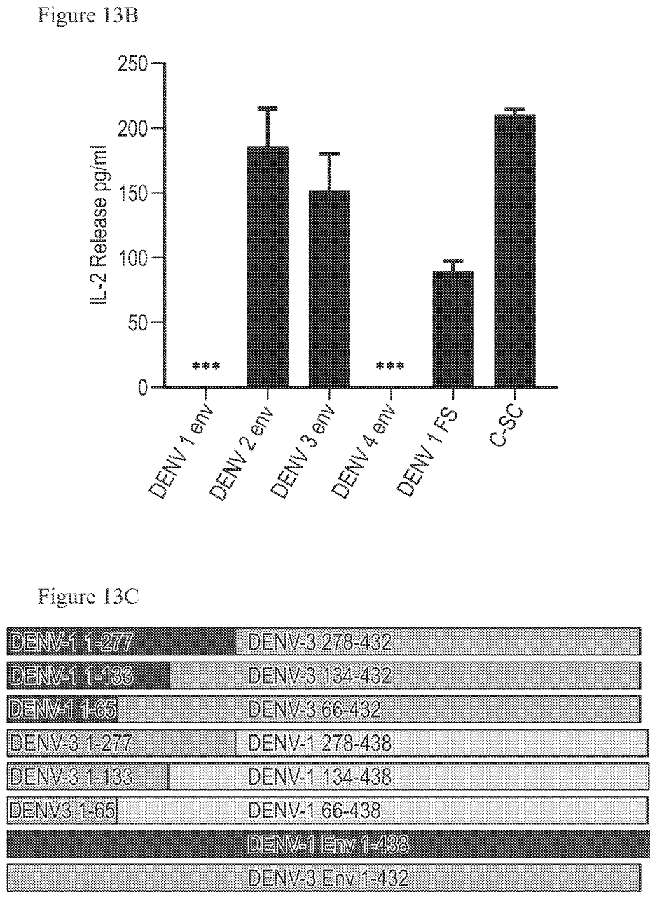

To determine if serotype induced TCR-mediated IL-2 differences was caused by a failure of DENV 1 and 4 to interact with the human T cells, viral binding to Jurkat cells was examined at 4° C. following 1 hr incubation with DENV equivalent RNA copies and found that DENV 1 had significantly less virus bound than DENV 2 or 3, and similar levels bound as DENV 4. Since DENV 1 and 4 inhibit TCR-mediated IL-2 release, the reduced binding does not explain the observed phenotype (FIG. 12A). Consistent with increased binding of DENV 2 and 3, more DENV 2 and 3 RNA entered cells when the temperature was raised to 37° C. for 1 hr after washing with PBS. While some of the virus detected in cells at 37° C. represented virus bound to the surface, trypsin treatment did not remove all DENV RNA (FIG. 12A). Thus, DENV does bind to and enter CD4+ Jurkat T cells. DENV 1 and 4 RNA levels increased intracellularly (FIG. 12B) and more DENV 1 RNA was released into culture supernatants over time in culture (FIG. 12C). In contrast, DENV 2 and 3 did not show further increases in intracellular viral RNA, and DENV 2, 3, and 4 released into culture supernatants did not increase over time (FIGS. 12B, 12C). No infectious DENV was detected in culture supernatants applied to Huh 7.5 cells or Vero cells for any serotype indicating that, despite binding, entry and possibly intracellular RNA replication, DENV infection of human T cell is abortive.

### DENV Envelope Protein Mediates TCR Inhibition

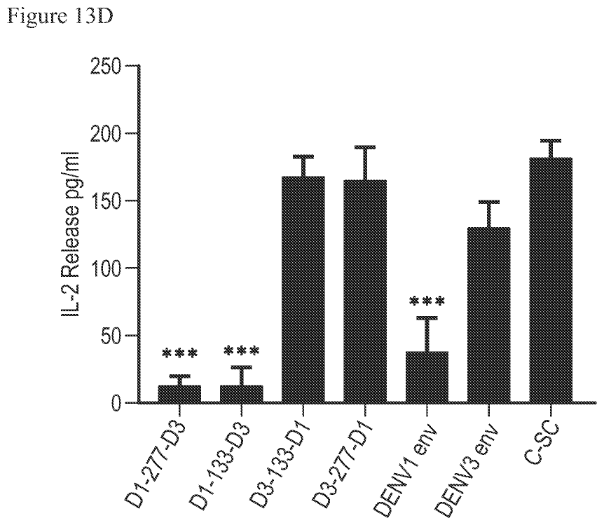

Since viral replication was not required for TCR inhibition, and DENV 1 and 4 particles were sufficient to inhibit TCR, it was postulated that viral structural proteins may be involved in TCR inhibition. DENV 1, 2, 3, 4 env proteins containing a C-terminal influenza hemagglutinin epitope were therefore expressed in Jurkat cells and 293T cells. Jurkat cells stably expressing GFP were generated as described for other viral env proteins. FIG. 13A schematically illustrates the four DENV env proteins stably expressed in the Jurkat and 293T cells. In addition, a frame-shift mutation (FS) was inserted at the start of the DENV 1 env coding region to provide a cell line that expresses the same RNA sequences without expressing the env protein. Expression of full-length env proteins was detected in 293T cells by immune blot using anti-HA antibodies. Deletion of the DENV 1, 2, 3, 4 env's C-terminal transmembrane domain resulted in detection of all four env proteins in Jurkat cells and all subsequent env constructs lack the transmembrane domain (data not shown).

Stimulating TCR with anti-CD3/CD28 in Jurkat cells expressing the DENV 1, 2, 3, or 4 env protein confirmed that DENV 1 and 4 envs inhibit TCR whereas DENV 2 and 3 env did not (FIG. 13B). This required protein expression, as Jurkat cells expressing the DV1 FS sequence did not result in TCR inhibition (FIG. 13B). In addition to the Jurkat expression system, we expressed the DENV 1, 2, 3, 4 and FS envelope sequences in 293T cells. As noted in methods, these cells were applied to the bottom of 24 well transwell plates and 2 days later, primary healthy donor PBMCs were added to the upper well of the plates and maintained for 2 additional days before addition of anti-CD3. Media was collected 16 hrs later and IL-2 release measured. Although the magnitude of inhibition was less than that observed in the Jurkat cells, DENV 1 and 4 envelopes inhibited primary PBMC TCR-mediated IL-2 release while DENV 2 and 3 did not (data not shown).

To characterize the env region involved in TCR inhibition, chimeric DENV1 and DENV3 envelopes were generated. FIG. 13C illustrates the DV1-DV3 chimeras generated. The N-terminal 133 amino acids of DENV1 were sufficient to maintain the TCR inhibitory phenotype when DV3 amino acids from 134 to the end (432) were expressed. Similarly, expression of DENV 1 amino acids 134 to 438 with DENV 31 to 133 did not inhibit TCR.

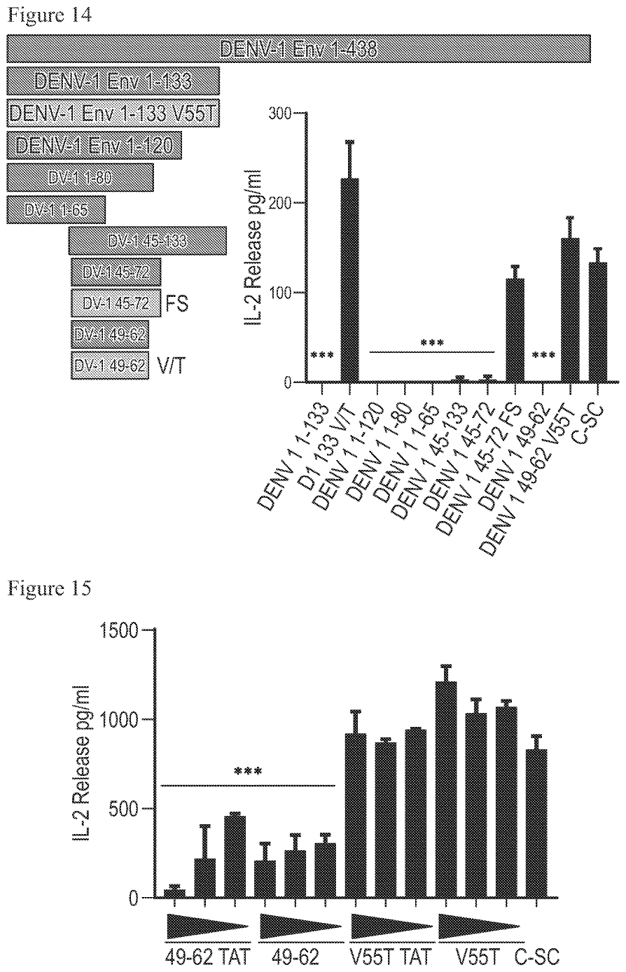

Examination of published DENV serotype envelope sequences in the first 133 amino acids of DENV 1, 2, 3, and 4 identified only 2 amino acids in DENV 1 (valine, serine) and in DENV 4 (leucine, serine) at positions 55 and 66 that are different from both DV 2 and 3 (threonine at both positions). Subsequent characterization of the TCR-inhibitory region of DENV 1 env protein was done by generating Jurkat cell lines expressing a series truncated proteins. FIG. 14 shows the schematic of full length ENV (1-438) and regions expressed. Cells expressing the 14 amino acid motif between 49 and 62 potently inhibited TCR-mediated IL-2 release following stimulation (FIG. 14). Further, mutation of the V55 in DENV 1 to the threonine present in DENV 2 and 3 abolished TCR inhibition when expressed in the 1-133 truncated protein, or in the 14 amino acid region (49-62) shown sufficient for TCR inhibition. Protein expression was required, as insertion of a frameshift into the start of the 14 aa region also abolished the TCR inhibition (FIG. 14).

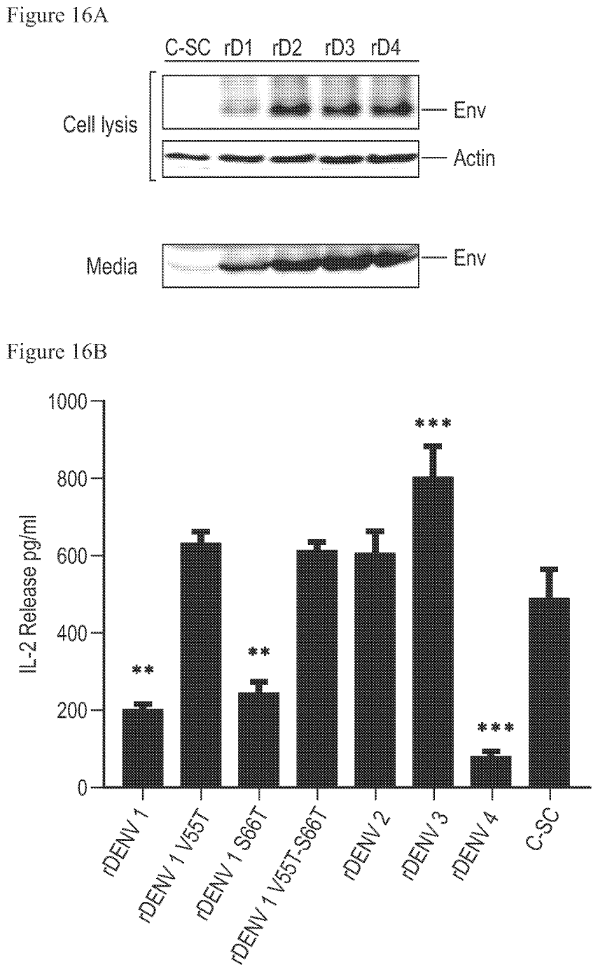

To determine if addition of the DENV 1 exogenous peptides could inhibit TCR, synthetic DENV 1 env sequences 49 to 62 peptides with and without an HIV TAT protein transduction domain were purchased (Sigma Aldrich, St. Louis, MO), as prior studies found that this basic amino acid sequence was required for human Pegivirus E2 peptides to enter the cell and inhibit TCR. This 14mer peptide was also synthesized in which the valine at position 55 was changed to threonine. Dose dependent inhibition of TCR-mediated IL-2 release was observed for the DENV 1 peptide with or without the TAT PTD domain, thus exogenous peptides are able to dampen T cell functions (FIG. 15). The critical residue 55 was confirmed as peptides containing V55T did not inhibit TCR.

### Reverse Genetics Confirm Role of DENV Env Mutations in TCR Phenotype and Viral Replication

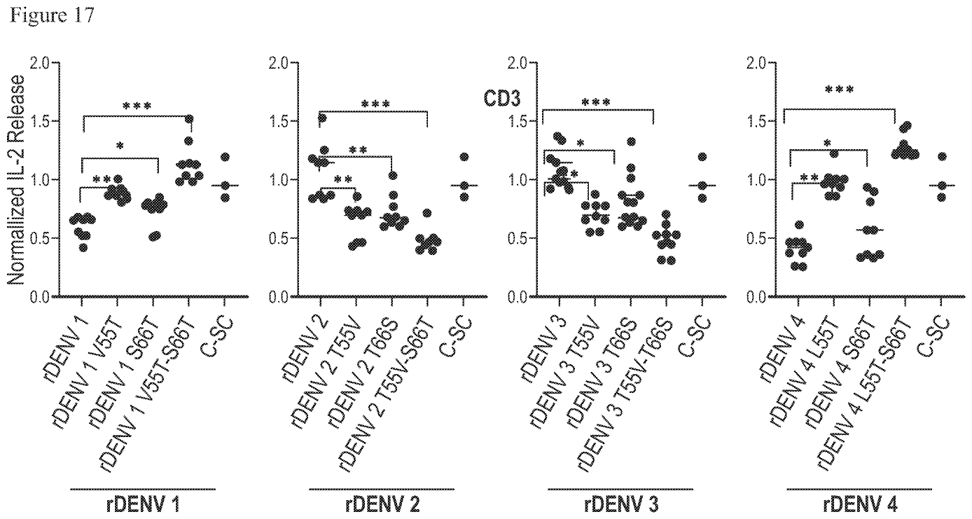

The development of CPER methods for generating infectious flavivirus clones markedly improves the ability to utilize reverse genetics in dengue virus. To determine if the V55T and/or the S66T amino acids identified in the Jurkat expression system were responsible for the differences in DENV 1 TCR inhibition, recombinant DENV 1, 2, 3, and 4 viruses were first generated using previously described CPER plasmids. Replication was confirmed in pass 2 virus by detecting dengue envelope protein expression intracellularly and in culture media supernatant fluids by immunoblot (FIG. 16A). rDENV 1 mutants were then generated containing single substitutions (V55T and S66T) or double substitutions and following verification of expression and mutation, and it was found that substituting the DENV 2 and 3 threonine for the DENV 1 valine restored TCR signaling as measured by IL-2 release following anti-CD3/CD28 stimulation in Jurkat cells. The T66S substitution did not restore TCR function.

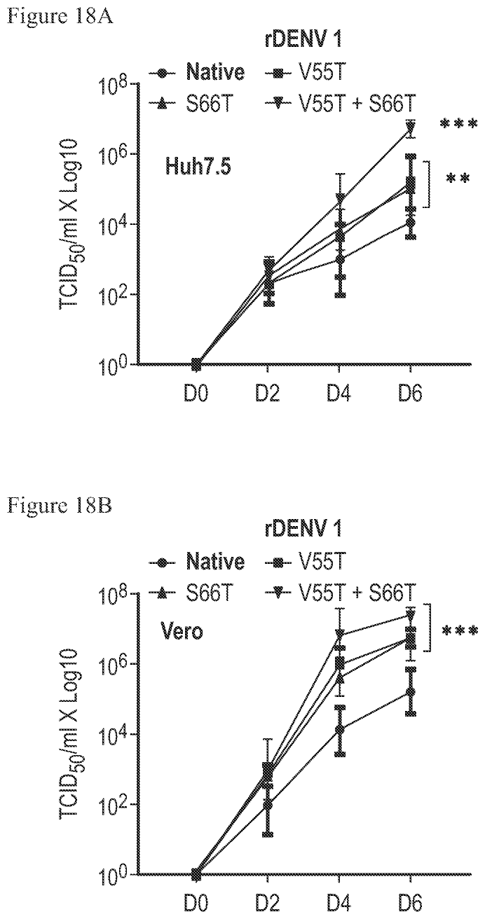

To further examine the role of DENV env amino acids 55 and 66 in TCR inhibition, rDENV 1, 2, 3, and 4 with single and double mutants were generated and, after confirming replication and sequence, the effects of each on TCR function in primary human PBMCs was examined. As in the Jurkat cells, rDENV1 V55T mutants reversed TCR inhibition (restored TCR functions) (FIG. 17). Of note, some reversal of TCR inhibition was observed in the T66S mutant as well. Confirming the importance of amino acid 55 in TCR inhibition, rDENV 4 L55T also restored TCR signaling, and as with rDENV 1, S66T substitutions had partial restoration of TCR function (FIG. 17). In both rDENV 1 and 4, TCR function was greater than control following incubation with virus containing both mutations. Mutation of rDENV 2 and 3 T55 to valine produced minimal (though significant) TCR inhibition and single mutations of T66 to serine produced similar levels of TCR in rDENV 2 but not 3. Mutation of both 55 and 66 resulted in significant TCR inhibition, similar to that observed for rDENV 1 and 4.

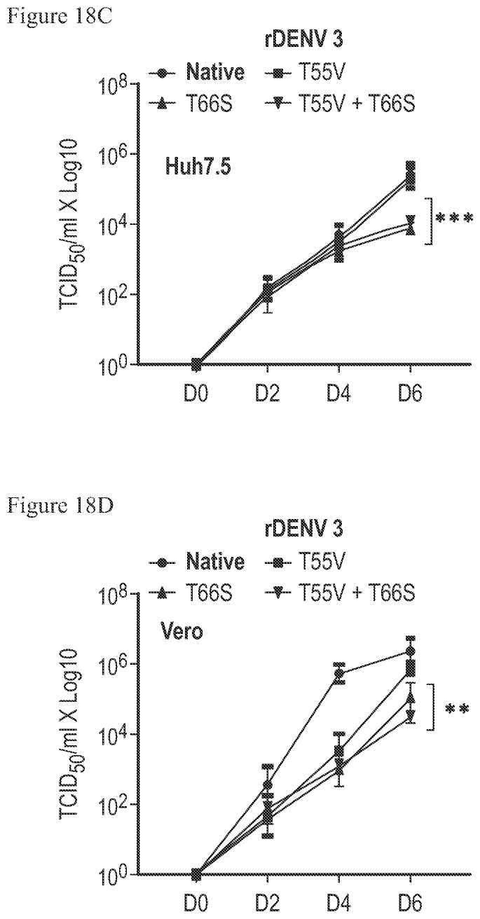

Unexpectedly, examination of the replication of the four rDENV 1 viruses found that the single amino acid mutants (55 and 66) increased viral titers in Huh 7.5 cells by 1.13 and 1.03 log₁₀ respectively, and by 2.69 log₁₀ in the double mutant (FIG. 18A). Similar increases in titer for the single mutants (1.54 and 1.56 log₁₀) and double mutant 2.11 log₁₀ were observed in Vero cells (FIG. 18B). The mutations in rDENV 3 had the opposite effect on replication, as the single (55 and 66) and double mutations decreased viral titers in Huh 7.5 cells by 1.06, 1.5 and 1.28 log₁₀ (FIG. 18C) and by 0.5, 1.23 and 1.89 log₁₀ in Vero cells. These data illustrate that DENV env amino acids 55 and 66 are not only involved in TCR signaling interference, but are critical for viral replication. Within Differential replication of isolates between DENV serotypes has been shown and mutations in DENV 4 in nonstructural proteins have been implicated; however, no specific env mutations have been identified that increase or decrease replication have been described. The data suggest that more efficient production of DENV 1 and 4 are possible with only one or two amino acid changes which can easily be introduced by CPER. The data show that rDENV 4 mutants replicate to significantly higher titers than wildtype virus, and that rDENV 2 mutants replicate to lower titers, consistent with the rDENV 1 and rDENV 2 data.

### SUMMARY

DENV2 and DENV3 cause more severe disease including hemorrhagic fever and dengue shock syndrome in people with prior dengue infection with a heterologous serotype. This is immunologically mediated. Some live attenuated vaccines have had similar problems. Mutating DENV2 and DENV3 to inhibit TCR reduces the risk of hyper-immune responses in vaccine recipients in live-attenuated vaccine use.

Subunit flavivirus envelope-based vaccines have not proven very immunogenic. This is likely influenced by the T cell inhibitory effects noted in DENV1 and DENV4. By removing these TCR inhibitory motifs in recombinant proteins, these envelope proteins are more immunogenic and thus more effective in subunit vaccine approaches.

Ability to make mutations at amino acid 55 to Threonine and amino acid 66 to Threonine of DENV 1 and 4 viral envelopes enhance replication and improve production.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A recombinant Dengue virus (DENV) envelope (env) protein,
wherein the DENV is serotype 1 and amino acid 55 is not a valine,
wherein the DENV is serotype 2 and amino acid 55 is a valine or a leucine,
wherein the DENV is serotype 3 and amino acid 55 is a valine or a leucine,
wherein the DENV is serotype 4 and amino acid 55 is not a leucine.

2. The recombinant DENV envelope protein of claim 1, wherein the DENV is serotype 1 or 4 and the amino acid at position 55 is threonine.

3. The recombinant DENV envelope protein of claim 1, wherein the DENV is serotype 1 or 4 and the protein further comprises a threonine at amino acid 66.

4. The recombinant DENV envelope protein of claim 1, wherein the DENV is serotype 1.

5. The recombinant DENV envelope protein of claim 1, wherein the DENV is serotype 4.

6. A composition comprising a protein comprising the recombinant DENV envelope protein of claim 1, and a pharmaceutically-acceptable, non-toxic vehicle, wherein the composition has enhanced antigenicity of DENV1 and DENV4 as compared to a non-recombinant DENV1 or DENV4 env protein, or wherein the composition has reduced inflammatory effects of DENV2 and DENV3 as compared to a non-recombinant DENV2 or DENV3 env protein.

7-8. (canceled)

9. The composition of claim 6, further comprising an effective amount of an immunological adjuvant.

10. The composition of claim 9, wherein the immunological adjuvant is selected from the group consisting of an aluminium salts Monophosphoryl lipid A (MPL) and aluminum salt, monophosphoryl lipid A (MPL) and aluminum salt, and an oil in water emulsion comprising squalene, monophosphoryl lipid A (MPL) and QS-21 and cytosine phosphoguanine (CpG).

11. (canceled)

12. The recombinant DENV envelope protein of claim 1, wherein the DENV is serotype 2.

13. The recombinant DENV envelope protein of claim 1, wherein the DENV is serotype 3.

14. The recombinant DENV envelope protein of claim 1, wherein the DENV is serotype 2 or serotype 3 and wherein the amino acid at position 55 is a valine.

15. The recombinant DENV envelope protein of claim 1, wherein the DENV is serotype 2 or serotype 3, and wherein the amino acid at position 55 is a leucine.

16. The recombinant DENV envelope protein of claim 1, wherein the DENV is serotype 2 or serotype 3, and wherein the protein further comprises a serine at amino acid 66.

17-21. (canceled)

22. A method of protecting a susceptible patient against DENV infection comprising administering an effective amount of the composition of claim 1 to the patient in need thereof.

23. The method of claim 22, wherein the composition is administered by intramuscular, intradermal, subcutaneous delivery, or via a mucosal surface.

24-28. (canceled)

29. A multivalent DENV composition comprising:
(a) a cell-adapted or attenuated DENV1 virus,
(b) a cell-adapted or attenuated DENV2 virus, wherein the envelope protein comprises a valine or a leucine at amino acid position 55,
(c) a cell-adapted or attenuated DENV3 virus, wherein the envelope protein comprises a valine or a leucine at amino acid position 55, and
(d) a cell-adapted or attenuated DENV4 virus.

30. The multivalent DENV composition of claim 29, wherein the env protein of (b) or (c) further comprises a serine at amino acid 66.

31. A multivalent DENV composition comprising:
(a) a subunit vaccine comprising a recombinant Dengue virus (DENV) envelope (env) protein, wherein the DENV is serotype 1 and amino acid 55 is not a valine,
(b) a subunit vaccine comprising a DENV2 env protein,
(c) a subunit vaccine comprising a DENV3 virus, and
(d) a subunit vaccine comprising a recombinant Dengue virus (DENV) envelope (env) protein, wherein the DENV is serotype 4 and amino acid 55 is not a leucine.

32. The multivalent DENV composition of claim 31, wherein in (a) the amino acid at position 55 is threonine and wherein in (d) the amino acid at position 55 is threonine.

33. The multivalent DENV composition of claim 31, wherein in (a) the amino acid at position 66 is threonine and wherein in (d) the amino acid at position 66 is threonine.

34. The recombinant Dengue virus (DENV) of claim 1, wherein the DENV replicates to a higher titer than wildtype virus.

35. The recombinant DENV of claim 34, wherein the DENV is serotype 1 and amino acid 55 of the env protein is not a valine, or wherein the DENV is serotype 4 and amino acid 55 of the env protein is not a leucine

36-37. (canceled)

38. The recombinant DENV of claim 34, wherein the titer is greater than 1.0 log₁₀ as compared to the wildtype virus titer.

39. (canceled)


SEQUENCE LISTING (31 sequences)

SEQ ID NO: 1 - protein, 135 aa, Dengue virus
MRCVGIGNRDFVEGLSGATWVDVVLEHGSCVTTMAKNKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYENL

SEQ ID NO: 2 - protein, 135 aa, Dengue virus; identical to SEQ ID NO: 1

SEQ ID NO: 3 - protein, 135 aa, Dengue virus
MRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENL

SEQ ID NO: 4 - protein, 135 aa, Dengue virus; identical to SEQ ID NO: 3

SEQ ID NO: 5 - protein, 135 aa, Dengue virus
MRCIGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKVVQHENL

SEQ ID NO: 6 - protein, 135 aa, Dengue virus; identical to SEQ ID NO: 5

SEQ ID NO: 7 - protein, 135 aa, Dengue virus
MRCVGVGNRDFVEGVSGGAWVDLVLEHGGCVTTMAQGKPTLDFELTKTTAKEVALLRTYCIEALISNITTATRCPTQGEPYLKEEQDQQYICRRDVVDRGWGNGCGLFGKGGVVTCAKFSCSGKITGNLVQIENL

SEQ ID NO: 8 - protein, 135 aa, Dengue virus; identical to SEQ ID NO: 7

SEQ ID NO: 9 - protein, 133 aa, Dengue virus
MRCVGIGNRDFVEGLSGATWVDVVLEHGSCVTTMAKNKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYE

SEQ ID NO: 10 - protein, 133 aa, Dengue virus
MRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPE

SEQ ID NO: 11 - protein, 133 aa, Dengue virus
MRCIGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKVVQHE

SEQ ID NO: 12 - protein, 133 aa, Dengue virus
MRCVGVGNRDFVEGVSGGAWVDLVLEHGGCVTTMAQGKPTLDFELTKTTAKEVALLRTYCIEALISNITTATRCPTQGEPYLKEEQDQQYICRRDVVDRGWGNGCGLFGKGGVVTCAKFSCSGKITGNLVQIE

SEQ ID NO: 13 - DNA, 24 bp, synthetic construct
  source 1..24: mol_type = other DNA
garagaccagagatcctgctgtct

SEQ ID NO: 14 - DNA, 21 bp, synthetic construct
  source 1..21: mol_type = other DNA
accattccattttctggcgtt

SEQ ID NO: 15 - DNA, 17 bp, synthetic construct
  source 1..17: mol_type = other DNA
  modified_base 1: mod_base = OTHER; note = 5' 6-Carboxyfluorescein modified nucleotide
  modified_base 17: mod_base = OTHER; note = 3' Iowa Black FQ modified nucleotide
agcatcattccaggcac

SEQ ID NO: 16 - DNA, 21 bp, synthetic construct
  source 1..21: mol_type = other DNA
gctggtgcaatctcacgtcta

SEQ ID NO: 17 - DNA, 17 bp, synthetic construct
  source 1..17: mol_type = other DNA
gcgcgaatcctgggttt

SEQ ID NO: 18 - DNA, 23 bp, synthetic construct
  source 1..23: mol_type = other DNA
  modified_base 1: mod_base = OTHER; note = 5' 6-Carboxyfluorescein modified nucleotide
  modified_base 23: mod_base = OTHER; note = 3' Iowa Black FQ modified nucleotide
atgcacccagagcggagaacgga

SEQ ID NO: 19 - DNA, 27 bp, synthetic construct
  source 1..27: mol_type = other DNA
aggatcccatgcgatgcgtgggaatag

SEQ ID NO: 20 - DNA, 27 bp, synthetic construct
  source 1..27: mol_type = other DNA
agcggccgctgcctgaaccatgactcc

SEQ ID NO: 21 - DNA, 28 bp, synthetic construct
  source 1..28: mol_type = other DNA
aggatcccatgcgttgcataggaatgtc

SEQ ID NO: 22 - DNA, 26 bp, synthetic construct
  source 1..26: mol_type = other DNA
agcggccgcggcctgcaccatgactc

SEQ ID NO: 23 - DNA, 28 bp, synthetic construct
  source 1..28: mol_type = other DNA
aggatcccatgagatgtgtaggagtagg

SEQ ID NO: 24 - DNA, 26 bp, synthetic construct
  source 1..26: mol_type = other DNA
agcggccgcagcttgcaccacggctc

SEQ ID NO: 25 - DNA, 27 bp, synthetic construct
  source 1..27: mol_type = other DNA
aggatcccatgcgatgcgtaggagtag

SEQ ID NO: 26 - DNA, 24 bp, synthetic construct
  source 1..24: mol_type = other DNA
agcggccgcttgaaccgtgaagcc

SEQ ID NO: 27 - protein, 172 aa, Dengue virus
ILLMLVTPSMAMRCVGIGNRDFVEGLSGATWVDVVLEHGSCVTTMAKNKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNETTEHGTT

SEQ ID NO: 28 - protein, 161 aa, Dengue virus
MRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKE

SEQ ID NO: 29 - protein, 159 aa, Dengue virus
MRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKVVQHENLKYTVIITVHTGDQHQVGNETQGVT

SEQ ID NO: 30 - protein, 161 aa, Dengue virus
MRCVGVGNRDFVEGVSGGAWVDLVLEHGGCVTTMAQGKPTLDFELTKTTAKEVALLRTYCIEALISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVT

SEQ ID NO: 31 - protein, 172 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 11: note = Any amino acid
  VARIANT 15: note = Any amino acid
  VARIANT 17: note = Any amino acid
  VARIANT 18: note = Any amino acid
  VARIANT 26: note = Any amino acid
  VARIANT 29: note = Any amino acid
  VARIANT 30: note = Any amino acid
  VARIANT 34: note = Any amino acid
  VARIANT 40: note = Any amino acid
  VARIANT 47: note = Any amino acid
  VARIANT 48: note = Any amino acid
  VARIANT 54: note = Any amino acid
  VARIANT 57: note = Any amino acid
  VARIANT 60: note = Any amino acid
  VARIANT 61: note = Any amino acid
  VARIANT 62: note = Any amino acid
  VARIANT 63: note = Any amino acid
  VARIANT 64: note = Any amino acid
  VARIANT 66: note = Any amino acid
  VARIANT 69: note = Any amino acid
  VARIANT 70: note = Any amino acid
  VARIANT 74: note = Any amino acid
  VARIANT 75: note = Any amino acid
  VARIANT 76: note = Any amino acid
  VARIANT 77: note = Any amino acid
  VARIANT 79: note = Any amino acid
  VARIANT 82: note = Any amino acid
  VARIANT 83: note = Any amino acid
  VARIANT 91: note = Any amino acid
  VARIANT 92: note = Any amino acid
  VARIANT 94: note = Any amino acid
  VARIANT 99: note = Any amino acid
  VARIANT 100: note = Any amino acid
  VARIANT 101: note = Any amino acid
  VARIANT 102: note = Any amino acid
  VARIANT 104: note = Any amino acid
  VARIANT 105: note = Any amino acid
  VARIANT 106: note = Any amino acid
  VARIANT 107: note = Any amino acid
  VARIANT 123: note = Any amino acid
  VARIANT 124: note = Any amino acid
  VARIANT 125: note = Any amino acid
  VARIANT 129: note = Any amino acid
  VARIANT 131: note = Any amino acid
  VARIANT 133: note = Any amino acid
  VARIANT 134: note = Any amino acid
  VARIANT 135: note = Any amino acid
  VARIANT 136: note = Any amino acid
  VARIANT 137: note = Any amino acid
  VARIANT 139: note = Any amino acid
  VARIANT 140: note = Any amino acid
  VARIANT 143: note = Any amino acid
  VARIANT 147: note = Any amino acid
  VARIANT 149: note = Any amino acid
  VARIANT 150: note = Any amino acid
  VARIANT 151: note = Any amino acid
  VARIANT 152: note = Any amino acid
  VARIANT 154: note = Any amino acid
  VARIANT 156: note = Any amino acid
  VARIANT 158: note = Any amino acid
  VARIANT 159: note = Any amino acid
  VARIANT 161: note = Any amino acid
  VARIANT 165: note = Any amino acid
  VARIANT 167: note = Any amino acid
  VARIANT 168: note = Any amino acid
  VARIANT 169: note = Any amino acid
  VARIANT 171: note = Any amino acid
  VARIANT 172: note = Any amino acid
XXXXXXXXXXXMRCXGXXNRDFVEGXSGXXWVDXVLEHGXCVTTMAXXKPTLDXELXKTXXXXXAXLRXXCIEXXXXNXTTXXRCPTQGEXXLXEEQDXXXXCXXXXVDRGWGNGCGLFGKGXXXTCAXFXCXXXXXGXXVQXENLXYXXXXTXHXGXXHXVGNXTXXXGXX